(19) United States
(12) Patent Application Publication
(10) Pub. No.: US 2011/0053248 A1
(43) Pub. Date: Mar. 3, 2011

(54) Serum-Free Virus Propagation Platform For A Virus Vaccine Candidate

(75) Inventors: Inn Yuk, Berkeley, CA (US); Wenlin Zeng, Union City, CA (US); Misa Sugui, Santa Clara, CA (US)

(73) Assignee: MEDIMMUNE, LLC, Gaithersburg, MD (US)

(21) Appl. No.: 12/445,950

(22) PCT Filed: Oct. 5, 2007
(86) PCT No.: PCT/US07/80610

§ 371 Date: Nov. 15, 2010

Related U.S. Application Data
(63) Continuation-in-part of application No. PCT/US2007/066037, filed on Apr. 5, 2007.
(60) Provisional application No. 60/862,550, filed on Oct. 23, 2006.

(51) Int. Cl. C12N 7/00; C12N 5/071

(52) U.S. Cl. 435/235.1; 435/364

(57) ABSTRACT

The invention relates to methods for propagating viruses. In particular, the invention provides optimized conditions for propagating viruses. Optimization of the following parameters are provided: lipid concentrates as supplements to the medium, temperature shift from pre-infection to post-infection, multiplicity of infection, direct bead-to-bead transfer and serum supplementation of pre-infection medium. In particular, the invention provides for the first time a method for propagating a virus by culturing cells that are infected with the virus in a medium comprising chemically defined lipid concentrate (CDLC). In another claim, the CDLC is added to medium that is substantially free of serum for culture of virus-infected cells.

### 1. INTRODUCTION

The invention relates to methods for propagating viruses. In particular, the invention provides optimized conditions for propagating viruses. Optimization of the following parameters are provided: lipid concentrates as supplements to the medium, temperature shift from pre-infection to post-infection, multiplicity of infection and serum supplementation of pre-infection medium. In particular, the invention provides for the first time a method for propagating a virus by culturing cells that are infected with the virus in a medium comprising chemically defined lipid concentrate (CDLC). In another embodiment, the CDLC is added to medium that is substantially free of serum for culture of virus-infected cells. In yet another embodiment, the invention provides for propagating a viral cell culture by a direct bead-to-bead transfer method.

### 2. BACKGROUND OF THE INVENTION

Human parainfluenza virus types 1-3 (hPIV1-3) and respiratory syncytial virus (RSV) and human metapneumovirus (hMPV) are non-segmented negative-strand RNA viruses of the paramyxovirus family. The Paramyxoviridae form a family within the order of Mononegavirales, consisting of the sub-families Paramyxovirinae and Pneumovirinae. Parainfluenza virus is a member of the Respirovirus genus (PIV1, PIV2 and PIV3) of the paramyxoviridae family. Human respiratory syncytial virus (hRSV), is a species of the Pneumovirus genus, and is the single most important cause of lower respiratory tract infections during infancy and early childhood worldwide (Domachowske, & Rosenberg, 1999, Clin Microbio Rev 12(2): 298-309). HMPV is a new member of the paramyxoviridae family with clinical symptoms reminiscent of those caused by hRSV infection, ranging from mild upper respiratory tract disease to severe bronchiolitis and pneumonia (Van Den Hoogen et al., 2001, Nature Medicine 7:719-724). The genomic organization of human metapneumovirus is described in van den Hoogen et al., 2002, Virology 295:119-132. Together, hPIV3 and RSV and hMPV are believed responsible for approximately one-third of all cases of pediatric respiratory diseases leading to hospitalization (Hall, 2001, N Eng J Med 344:1917-1927).

2.1 PIV Infections

Parainfluenza viral infection (PIV) results in serious respiratory tract disease in infants and children. (Tao et al., 1999, Vaccine 17: 1100-08). Infectious parainfluenza viral infections account for approximately 20% of all hospitalizations of pediatric patients suffering from respiratory tract infections worldwide. Id.

PIV is made up of two structural modules: (1) an internal ribonucleoprotein core, or nucleocapsid, containing the viral genome, and (2) an outer, roughly spherical lipoprotein envelope. Its genome is a single strand of negative sense RNA, approximately 15,456 nucleotides in length, encoding at least eight polypeptides. These proteins include, but are not limited to, the nucleocapsid structural protein (NP, NC, or N depending on the genera), the phosphoprotein (P), the matrix protein (M), the fusion glycoprotein (F), the hemagglutinin-neuraminidase glycoprotein (HN), the large polymerase protein (L), and the C and D proteins of unknown function. Id.

The parainfluenza nucleocapsid protein (NP, NC, or N) consists of two domains within each protein unit including an amino-terminal domain, comprising about two-thirds of the molecule, which interacts directly with the RNA, and a carboxyl-terminal domain, which lies on the surface of the assembled nucleocapsid. A hinge is thought to exist at the junction of these two domains thereby imparting some flexibility to this protein (see Fields et al. (ed.), 1991, Fundamental Virology, Second Edition, Raven Press, New York, incorporated by reference herein in its entirety). The matrix protein (M), is apparently involved with viral assembly and interacts with both the viral membrane as well as the nucleocapsid proteins. The phosphoprotein (P), which is subject to phosphorylation, is thought to play a regulatory role in transcription, and may also be involved in methylation, phosphorylation and polyadenylation. The fusion glycoprotein (F) interacts with the viral membrane and is first produced as an inactive precursor, then cleaved post-translationally to produce two disulfide linked polypeptides. The active F protein is also involved in penetration of the parainfluenza virion into host cells by facilitating fusion of the viral envelope with the host cell plasma membrane. Id. The glycoprotein, hemagglutinin-neuraminidase (FIN), protrudes from the envelope allowing the virus to contain both hemagglutinin and neuraminidase activities. FIN is strongly hydrophobic at its amino terminal which functions to anchor the FIN protein into the lipid bilayer. Id. Finally, the large polymerase protein (L) plays an important role in both transcription and replication. Id.

2.2 RSV Infections

Respiratory syncytial virus (RSV) is the leading cause of serious lower respiratory tract disease in infants and children (Feigen et al., eds., 1987, In: Textbook of Pediatric Infectious Diseases, W B Saunders, Philadelphia at pages 1653-1675; New Vaccine Development, Establishing Priorities, Vol. 1, 1985, National Academy Press, Washington D.C. at pages 397-409; and Ruuskanen et al., 1993, Curr Probl Pediatr 23:50-79). The yearly epidemic nature of RSV infection is evident worldwide, but the incidence and severity of RSV disease in a given season vary by region (Hall, 1993, Contemp Pediatr 10:92-110). In temperate regions of the northern hemisphere, it usually begins in late fall and ends in late spring. Primary RSV infection occurs most often in children from 6 weeks to 2 years of age and uncommonly in the first 4 weeks of life during nosocomial epidemics (Hall et al., 1979, New Engl J Med 300:393-396). Children at increased risk for RSV infection include, but are not limited to, preterm infants (Hall et al., 1979, New Engl J Med 300:393-396) and children with bronchopulmonary dysplasia (Groothuis et al., 1988, Pediatrics 82:199-203), congenital heart disease (MacDonald et al., New Engl J Med 307:397-400), congenital or acquired immunodeficiency (Ogra et al., 1988, Pediatr Infect Dis J 7:246-249; and Pohl et al., 1992, J Infect Dis 165:166-169), and cystic fibrosis (Abman et al., 1988, J Pediatr 113:826-830). The fatality rate in infants with heart or lung disease who are hospitalized with RSV infection is 3%-4% (Navas et al., 1992, J Pediatr 121:348-354).

RSV infects adults as well as infants and children. In healthy adults, RSV causes predominantly upper respiratory tract disease. It has recently become evident that some adults, especially the elderly, have symptomatic RSV infections more frequently than had been previously reported (Evans, A. S., eds., 1989, Viral Infections of Humans. Epidemiology and Control, 3rd ed., Plenum Medical Book, New York at pages 525-544). Several epidemics also have been reported among nursing home patients and institutionalized young adults (Falsey, A. R., 1991, Infect Control Hosp Epidemiol 12:602-608; and Garvie et al., 1980, Br Med J 281:1253-1254). Finally, RSV may cause serious disease in immunosuppressed persons, particularly bone marrow transplant patients (Hertz et al., 1989, Medicine 68:269-281).

Treatment options for established RSV disease are limited. Severe RSV disease of the lower respiratory tract often requires considerable supportive care, including administration of humidified oxygen and respiratory assistance (Fields et al., eds, 1990, Fields Virology, 2nd ed., Vol. 1, Raven Press, New York at pages 1045-1072).

While a vaccine might prevent RSV infection, and/or RSV-related disease, no vaccine is yet licensed for this indication. A major obstacle to vaccine development is safety. A formalin-inactivated vaccine, though immunogenic, unexpectedly caused a higher and more severe incidence of lower respiratory tract disease due to RSV in immunized infants than in infants immunized with a similarly prepared trivalent parainfluenza vaccine (Kim et al., 1969, Am J Epidemiol 89:422-434; and Kapikian et al., 1969, Am J Epidemiol 89:405-421). Several candidate RSV vaccines have been abandoned and others are under development (Murphy et al., 1994, Virus Res 32:13-36), but even if safety issues are resolved, vaccine efficacy must also be improved. A number of problems remain to be solved. Immunization would be required in the immediate neonatal period since the peak incidence of lower respiratory tract disease occurs at 2-5 months of age. The immaturity of the neonatal immune response together with high titers of maternally acquired RSV antibody may be expected to reduce vaccine immunogenicity in the neonatal period (Murphy et al., 1988, J Virol 62:3907-3910; and Murphy et al., 1991, Vaccine 9:185-189). Finally, primary RSV infection and disease do not protect well against subsequent RSV disease (Henderson et al., 1979, New Engl J Med 300:530-534).

Currently, the only approved approach to prophylaxis of RSV disease is passive immunization. Initial evidence suggesting a protective role for IgG was obtained from observations involving maternal antibody in ferrets (Prince, G. A., Ph.D. diss., University of California, Los Angeles, 1975) and humans (Lambrecht et al., 1976, J Infect. Dis 134:211-217; and Glezen et al., 1981, J Pediatr 98:708-715). Hemming et al. (Morell et al., eds., 1986, Clinical Use of Intravenous Immunoglobulins, Academic Press, London at pages 285-294) recognized the possible utility of RSV antibody in treatment or prevention of RSV infection during studies involving the pharmacokinetics of an intravenous immune globulin (IVIG) in newborns suspected of having neonatal sepsis. In this study, it was noted that one infant, whose respiratory secretions yielded RSV, recovered rapidly after IVIG infusion. Subsequent analysis of the IVIG lot revealed an unusually high titer of RSV neutralizing antibody. This same group of investigators then examined the ability of hyperimmune serum or immune globulin, enriched for RSV neutralizing antibody, to protect cotton rats and primates against RSV infection (Prince et al., 1985, Virus Res 3:193-206; Prince et al., 1990, J Virol 64:3091-3092; Hemming et al., 1985, J Infect Dis 152:1083-1087; Prince et al., 1983, Infect Immun 42:81-87; and Prince et al., 1985, J Virol 55:517-520). Results of these studies indicate that IVIG may be used to prevent RSV infection, in addition to treating or preventing RSV-related disorders.

A humanized antibody directed to an epitope in the A antigenic site of the F protein of RSV, SYNAGIS®, is approved for intramuscular administration to pediatric patients for of serious lower respiratory tract disease caused by RSV at recommended monthly doses of 15 mg/kg of body weight throughout the RSV season (November through April in the northern hemisphere). SYNAGIS® is a composite of human (95%) and murine (5%) antibody sequences. See, Johnson et al., 1997, J. Infect. Diseases 176:1215-1224 and U.S. Pat. No. 5,824,307, the entire contents of which are incorporated herein by reference. The human heavy chain sequence was derived from the constant domains of human IgG₁ and the variable framework regions of the VH genes or Cor (Press et al., 1970, Biochem. J. 117:641-660) and Cess (Takashi et al., 1984, Proc. Natl. Acad. Sci. USA 81:194-198). The human light chain sequence was derived from the constant domain of Cκ and the variable framework regions of the VL gene K104 with Jκ-4 (Bentley et al., 1980, Nature 288:5194-5198). The murine sequences derived from a murine monoclonal antibody, Mab 1129 (Beeler et al., 1989, J. Virology 63:2941-2950), in a process which involved the grafting of the murine complementarity determining regions into the human antibody frameworks.

2.3 HMPV Infections

Recently, a new member of the Paramyxoviridae family has been isolated from 28 children with clinical symptoms reminiscent of those caused by human respiratory syncytial virus (“hRSV”) infection, ranging from mild upper respiratory tract disease to severe bronchiolitis and pneumonia (Van Den Hoogen et al., 2001, Nature Medicine 7:719-724). The new virus was named human metapneumovirus (hMPV) based on sequence homology and gene constellation. The study further showed that by the age of five years virtually all children in the Netherlands have been exposed to hMPV and that the virus has been circulating in humans for at least half a century. Additionally, the seasonality of the infection is similar to RSV, peaking in the winter months (Robinson, 2005, J. Med. Virol. 76:98-105; Williams, 2004, New Engl. J. Med. 350:443-450). However, unlike RSV, hMPV can be isolated year-round, albeit at a lower rate (Robinson, 2005, J. Med. Virol. 76:98-105; Williams, 2004, New Engl. J. Med. 350:443-450). Risk factors for hMPV infection are also similar to those found for RSV. Highest incidence of infection with human metapneumovirus has been found in young children, in the elderly and immunocompromised humans. Infection with human metapneumovirus is a significant burden of disease in at-risk premature infants, chronic lung disease of prematurity, congestive heart disease, and immunodeficiency (Robinson, 2005, J. Med. Virol. 76:98-105; Williams, 2004, New Engl. J. Med. 350:443-450). Human metapneumovirus has recently been isolated from patients in North America (Peret et al., 2002, J. Infect. Diseases 185:1660-1663).

The genomic organization of human metapneumovirus is described in van den Hoogen et al., 2002, Virology 295:119-132. Phylogenetic analysis divides the hMPV strains into two genetic clusters, designated subgroups A and B that are distinct from APV viruses (Bastien et al 2003a and b; Biacchesi et al, 2003; Peret et al 2002 and 2004; van den Hoogen, 2002). Within these subgroups, hMPV can be further subdivided into A1, A2, B1, and B2 subtypes (van den Hoogen, 2003).

hMPV shares a similar genetic structure to RSV but lacks the non-structural genes found in RSV (van den Hoogen, 2002, Virology. 295:119-132). Both viruses code for similar surface proteins that are defined as the surface glycoprotein (G) protein and the fusion (F) protein. Based upon differences between the amino acid sequences of the G and F proteins, both RSV and hMPV have been subdivided into A and B groups. However, in hMPV there is a further bifurcation of A and B subgroups into A1, A2, B1, and B2 groupings (Boivin, 2004, Emerg. Infect. Dis. 10:1154-1157, 25). For both RSV and hMPV viruses, the sequences of the G proteins display a wide variance between subgroups; with hMPV the G protein has only 30% identity between A and B subgroups. For both RSV and hMPV the F protein is more conserved; across the known hMPV isolates the F protein amino acid sequence is 95% conserved (Biacchesi, 2003, Virology 315:1-9; Boivin, 2004, Emerg. Infect. Dis. 10:1154-1157; van den Hoogen, 2004, Emerg. Infect. Dis. 10:658-666). Despite the similarities in structure of the viruses, the F proteins of hMPV and RSV share only a 33% amino acid sequence identity and antisera generated against either RSV or hMPV do not neutralize across the pneumoviridae group (Wyde, 2003, Antiviral Research. 60:51-59). With RSV a single monoclonal antibody directed at the fusion (F) protein can prevent severe lower respiratory tract RSV infection. Similarly, because of the high level of sequence conservation of the F protein across all the hMPV subgroups, this protein is likely to be the preferred antigenic target for the generation of cross-subgroup neutralizing antibodies.

Human metapneumovirus is related to avian metapneumovirus. For example, the F protein of hMPV is highly homologous to the F protein of avian pneumonovirus (“APV”). Alignment of the human metapneumoviral F protein with the F protein of an avian pneumovirus isolated from Mallard Duck shows 85.6% identity in the ectodomain. Alignment of the human metapneumoviral F protein with the F protein of an avian pneumovirus isolated from Turkey (subgroup B) shows 75% identity in the ectodomain. See, e.g., co-owned and co-pending Provisional Application No. 60/358,934, entitled “Recombinant Parainfluenza Virus Expression Systems and Vaccines Comprising Heterologous Antigens Derived from Metapneumovirus,” filed on Feb. 21, 2002, by Haller and Tang, which is incorporated herein by reference in its entirety.

Based upon the recent work with hMPV, hMPV likewise appears to be a significant factor in human, particularly, juvenile respiratory disease.

Thus, despite the fact that a significant portion of human respiratory diseases is caused by hPIV3 and RSV and hMPV infections, vaccines against these viruses remain unavailable (Tang et al., 2004, J Virol 78:11198-11207).

2.4 Chimeric Viruses

It was reported (Haller et al., 2000, J Virol 74:11626-11635) that a recombinant bovine PIV3 vector was constructed by replacing the fusion (F) and hemagglutinin-neuraminidase (HN) glycoprotein genes in bovine PIV3 with the human PIV3 and NH genes, respectively. A later publication (Tang et al., 2003, J Virol 77:10819-10828) described an insertion of the RSV F gene into the bovine-human PIV3 vector backbone to generate a chimeric virus that expressed the RSV F protein. The chimeric virus was named MEDI-534. It functioned as a live, attenuated, bivalent vaccine in animal studies: hamsters and non-human primates immunized with MEDI-534 demonstrated protection from challenge with RSV and hPIV3, and the animals produced RSV-neutralizing and hPIV3 hemagglutination-inhibiting serum antibodies (Tang et al., 2003, supra; Tang et al., 2004, supra).

In light of the promising immunogenecity and the protection results in preclinical animal models, the replication of different viruses closely related to MEDI-534 was performed in different permissive mammalian cell lines (Haller et al., 2003, J Gen Virol 84:2153-2162). In all instances Vero cells generated the highest virus titers. It was shown that the MEDI-534 virus maintained the RSV F gene inserts stably for up to 10 serial passages in Vero cells (Tang et al., 2003, supra).

2.5 Propagating Viruses

The present invention relates to methods for propagating viruses. The conditions for the propagation of virus are optimized in order to produce a robust and high-yielding cell culture which would be beneficial, e.g., for manufacture of virus vaccine candidates of the invention.

### Serum-Free Medium

In view of growing concerns about human exposure to infectious agents such as transmissible spongiform encephalopathies (Asher, 1999, Dev Biol Stand 100: 103-118; Galbraith, 2002, Cytotechnology 39: 117-124) and adventitious viruses (Erickson et al., 1989, Dev Biol Stand 70: 59-66), regulatory authorities in the United States (Food and Drug Administration) and in Europe (European Medicine Evaluation Agency) encouraged biologics manufacturers to reduce or eliminate the use of substances of animal origin in their production processes (Castle and Robertson, 1999, Dev Biol Stand 99: 191-196).

### Thermal Sensitivity

Thermal sensitivity has been observed in virus systems: the cultivation of retrovirus packaging cells at 32° C. instead of 37° C. increased the yield of infectious virus particles by a maximum of 2- to 15-fold (Kaptein et al., 1997, Gene Therapy 4: 172-176; Kotani et al., 1994 Hum Gene Ther 5: 19-28; Lee et al., 1996, Appl Microbiol Biotechnol 45: 477-483).

### Cell Density Effect

The “cell density effect” has been observed for adenovirus production in batch cultures: specific virus productivity decreased with increasing cell density at the time of infection (Henry et al., 2004, Biotechnol Bioeng 86: 765-774; Nadeau and Kamen, 2003, Biotechnol Adv 20: 475-489). The successes in using perfusion cultures to overcome the “cell density effect” in adenovirus production (Henry et al., 2004, Biotechnol Bioeng 86: 765-774; Yuk et al., 2004 Biotechnol Bioeng 86: 637-642), compared with the failures encountered using conventional fed-batch strategies (Nadeau et al, 2002; Yuk et al., 2004 Biotechnol Bioeng 86: 637-642), allude to the presence of one or more unidentified inhibitors that accumulate under batch and fed-batch conditions.

### 3. SUMMARY OF THE INVENTION

The present invention relates to a method for propagating viruses. In certain embodiments, the invention provides a method for propagating negative strand RNA viruses. In particular, the invention provides a method for propagating non-segmented, negative strand RNA viruses, such as paramyxoviruses. The invention specifically provides a method for propagating parainfluenza virus (PIV) and respiratory syncytial virus (RSV) and metapneumovirus (MPV). Even more specifically, the invention provides a method for propagating PIV with human and bovine sequences. In one embodiment, chimeric human/bovine PIV expressing RSV nucleotide sequences is propagated using the methods of the invention.

In particular, the invention provides a method for propagating a virus by culturing cells that are infected with the virus in a medium comprising chemically defined lipid concentrate (CDLC). In a specific embodiment, the CDLC-supplemented medium is substantially free of serum. In certain embodiments, the medium is a serum-free medium, such as OptiPRO™ SFM or VP-SFM™ or SFM4 MegaVir™ (SFM4MV) or Ex-Cell Vero™ or Williams' E medium. In certain embodiments, the cells used are Vero cells. In another embodiment, the CDLC-supplemented medium contains a maximum serum concentration of 0.5% v/v. In a specific embodiment, the CDLC comprises one or more of Pluronic F-68, Ethyl Alcohol, Cholesterol, Tween 80, DL-alpha-Tocopherol Acetate, Stearic Acid, Myristic Acid, Oleic Acid, Linoleic Acid, Palmitic Acid, Palmitoleic Acid, Arachidonic Acid, and Linolenic Acid.

In another embodiment, the invention provides a method for propagating a virus by culturing cells infected by a virus in a serum-free medium. Contemplated serum-free media include, for example, VP-SFM™ or SM4 MegaVir™, OptiPRO™ SFM or Ex-Cell Vero™ or Williams' E medium. In certain embodiments, the cells used are Vero cells. In another embodiment, the cells used are serum-free adapted Vero cells.

The present invention provides further optimized conditions for propagating viruses. In one embodiment, the cells are cultured at a first temperature before infection with the virus and at a second temperature after infection with the virus, wherein the second temperature is lower than the first temperature. In a specific embodiment, the cells are cultured at about 37° C. before infection with the virus, i.e., pre-infection, and from about 29° C. to about 37° C. after infection with the virus, i.e., post-infection. In another embodiment, the cells are cultured at about 33° C. after infection with the virus. In another embodiment, the cells are cultured at about 30° C. after infection with the virus.

In one embodiment, the seeding density of the virus ranges from 1e5 to 2e5 cells/cm². In another embodiment, the seeding density is 2.1 e4 to 2.9 e4 cells/cm². In another embodiment, the seeding density is 2.4 e4 cells/cm². In yet another embodiment, the seeding density of the virus is 2e5 cells/cm².

In another embodiment, the cells are cultured with a virus at a multiplicity of infection (MOI) ranging from about 0.0001 to about 0.1. In yet another embodiment, the cells are cultured at a MOI ranging from about 0.001 to about 0.01.

In yet another embodiment, the cells are cultured in the presence of serum before infection with the virus. In an embodiment, the pre-infection medium comprises fetal bovine serum.

In one embodiment, the point of infection is 3 or 4 days post-seeding (dps). In one embodiment, the post-infection time ranges from 4 to 11 days. In another embodiment, the post-infection time is about 5 days or about 6 days or about 8 days. In yet another embodiment, the point of infection is when the cells reach ≧1e6 cells/cm².

In one embodiment, the viral cell cultures of the invention are grown in suspension culture in a bioreactor either in a batch process or in a fed-batch process. In another embodiment, the viral cell cultures of the invention are grown on microcarrier beads in said bioreactors. In a specific embodiment, the viral cell cultures of the invention are grown in suspension culture in a single use bioreactor (SUB). In another embodiment, the viral cell cultures of the invention are grown on microcarrier beads in a SUB.

In one embodiment, the viral titer as a result of the methods and compositions of the invention, is at least 5 log₁₀ TCID₅₀/ml, at least 6 log₁₀ TCID₅₀/ml, at least 7 log₁₀ TCID₅₀/ml, at least 8 log₁₀ TCID₅₀/ml, at least 9 log₁₀ TCID₅₀/ml, at least 10 log₁₀ TCID₅₀/ml.

The methods of the invention can also be used when rescuing virus from recombinant viral genomes.

3.1 Conventions and Abbreviations
ADCF animal derived component free
CDLC chemically defined lipid concentrate
Dpi days post-infection
Dps days post-seeding
ΔM2-2 Recombinant live attenuated RSV virus with a deletion of the M2-2 open reading frame (Jin et al., Vaccine, 2003 pp. 3647-52)
ΔNS-1 Recombinant live attenuated RSV virus with a deletion of the NS-1 open reading frame (Jin et al., Vaccine, 2003 pp. 3647-52)
F fusion
FBS fetal bovine serum
HMPV Human metapneumovirus
hPIV1-3 human parainfluenza virus types 1, 2 or 3
HN hemagglutinin-neuraminidase
MEDI-534 chimeric bovine/human parainfluenza virus type 3/respiratory syncytial virus (Tang et al., 2003 J Virol 77: 10819-1828)
MEDI-559 MEDI-559 is a live, attenuated RSV vaccine, termed rA2 cp248/404/1030ΔSH. (Karron et al., JID vol 191 p. 1093 (2005))
MOI multiplicity of infection
PIV3 parainfluenza virus type 3
Propagating increasing the number of virus particles
RB roller bottle
MEDI-560 or Recombinant cold passaged (45 cycles) of wt
rcp45 hPIV3 HPIV3 (Karron et al., Pediatr. Inf. Dis. J. 2003, 22:394-405)
RSV respiratory syncytial virus
SFM serum-free media
SUB Single use bioreactor
TCID₅₀ 50% tissue culture infectious dose
Vero African green monkey kidney cell line
v/v volume per volume ratio

### 4. DESCRIPTION OF FIGURES

FIG. 1. Virus production profiles at different MOIs. Duplicate T-75 flasks of serum-free Vero cells were infected three days post-seeding with MEDI-534 at MOI of 0.1, 0.01, 0.001, 0.0001 or 0.00001. Cultures were incubated at 37° C. pre- and post-infection.

FIG. 2a. Effects of time of infection and post-infection temperature on infectious virus titers. Serum-free Vero cultures were infected with MEDI-534 using MOI 0.001 at three days post-seeding (0.6×10⁷ cells/flask). Duplicate T-75 flasks were incubated at either 33° C. or 37° C. post-infection.

FIG. 2b. Effects of time of infection and post-infection temperature on infectious virus titers. Serum-free Vero cultures were infected with MEDI-534 using MOI 0.001 at five days post-seeding (0.6×10⁷ cells/flask). Duplicate T-75 flasks were incubated at either 33° C. or 37° C. post-infection.

FIG. 3. Virus production profiles of RB cultures titrated with FBS pre-infection. Vero cells were seeded in one of the following media in triplicate RBs: OptiPRO™ SFM, OptiPRO™ SFM+0.5% (v/v) FBS, and OptiPRO™+2% (v/v) FBS. Three days post-seeding, one RB in each condition was trypsinized for cell counting: OptiPRO™ SFM (1.9×10⁷ cells/flask), OptiPRO™ SFM+0.5% (v/v) FBS (9.3×10⁷ cells/flask) and OptiPRO™+2% (v/v) FBS (10.4×10⁷ cells/flask). The remaining 2×3 RBs were infected with MEDI-534 at MOI 0.001.

FIG. 4a. Comparison of cell yield in different pre-infection media and supplements. Vero cells were seeded in one of the following five media at four RBs per condition: (1) OptiPRO™ SFM, (2) OptiPRO™ SFM+1% (v/v) CDLC, (3) OptiPRO™+0.5% (v/v) FBS, (4) VP-SFM and (5) VP-SFM+1% (v/v) CDLC. Three days post-seeding, two RBs per condition were used for cell counts. The remaining duplicate sets of RBs were infected with MEDI-534 at MOI 0.001.

FIG. 4b. Comparison of virus production in different pre-infection media and supplements. Vero cells were seeded in one of the following five media at four RBs per condition: (1) OptiPRO™ SFM, (2) OptiPRO™ SFM+1% (v/v) CDLC, (3) OptiPRO™+0.5% (v/v) FBS, (4) VP-SFM and (5) VP-SFM+1% (v/v) CDLC. Three days post-seeding, two RBs per condition were used for cell counts. The remaining duplicate sets of RBs were infected with MEDI-534 at MOI 0.001.

FIG. 5a. Comparison of cell growth in RBs using different pre-infection media. Vero cells were seeded in either OptiPRO™+0.5% (v/v) FBS or VP-SFM+1% (v/v) CDLC. To generate the growth curves, duplicate RBs in each condition were counted daily.

FIG. 5b. Comparison of virus production in RBs using different pre-infection media. Vero cells were seeded in either OptiPRO™+0.5% (v/v) FBS or VP-SFM+1% (v/v) CDLC. To generate the virus production profiles, duplicate RB cultures in the two different pre-infection media were infected with MEDI-534 three days post-seeding. The infected cultures were sampled daily from 2-7 days post-infection.

FIG. 6a. Comparison of cell yield in RB cultures titrated with CDLC pre-infection. Vero cells were seeded in serum-free growth medium (VP-SFM) supplemented with CDLC at three different concentrations in duplicates. Four days post-seeding, the cultures were trypsinized for cell counting and passaged in the VP-SFM containing CDLC added at three different concentrations (in replicates of four). On the third day post-seeding, duplicate RBs per condition were counted, and the remaining duplicate sets of RBs were infected with MEDI-534.

FIG. 6b. Comparison of virus production in RB cultures titrated with CDLC pre-infection. Vero cells were seeded in serum-free growth medium (VP-SFM) supplemented with CDLC at three different concentrations in duplicates. Four days post-seeding, the cultures were trypsinized for cell counting and passaged in the VP-SFM containing CDLC added at three different concentrations (in replicates of four). On the third day post-seeding, duplicate RBs per condition were counted, and the remaining duplicate sets of RBs were infected with MEDI-534.

FIG. 7. Comparison of MEDI-534 production in different post-infection media. RB cultures were inoculated in VP-SFM+1% (v/v) CDLC. Three days post-seeding, duplicate RBs were infected at MOI 0.001 with MEDI-534 in one of the following SFM: VP-SFM+1% CDLC, VP-SFM and WME.

FIG. 8a. Comparison of cell growth in microcarrier cultures using different pre-infection media. Vero cells were seeded in duplicate spinner flasks containing 2 g/L Cytodex™ 1 in either OptiPRO™+0.5% (v/v) FBS or VP-SFM+1% (v/v) CDLC. To generate the growth curves, samples were taken daily from the uninfected flasks for nuclei counts.

FIG. 8b. Comparison of virus production in microcarrier cultures using different pre-infection media. Vero cells were seeded in duplicate spinner flasks containing 2 g/L Cytodex™ 1 in either OptiPRO™+0.5% (v/v) FBS or VP-SFM+1% (v/v) CDLC. To generate the virus production profiles, duplicate flasks in each pre-infection media were infected five days post-seeding with MEDI-534.

FIG. 9a. Comparison of pre-infection Vero cell growth in bioreactors controlled at different pHs. Vero cells were seeded in bioreactors containing 2 g/L Cytodex™ 1 in VP-SFM+1% (v/v) CDLC maintained at pH 7.0, ph 7.2 or pH 7.4. Samples were taken daily from the bioreactors for nuclei counts pre-infection.

FIG. 9b. Comparison of virus production in bioreactors controlled at different pHs. Vero cells were seeded in bioreactors containing 2 g/L Cytodex™ 1 in VP-SFM+1% (v/v) CDLC maintained at pH 7.0, ph 7.2 or pH 7.4. Four days post-seeding, the bioreactor cultures were infected with MEDI-534.

FIG. 10. Effects of agitation rate on Vero cell growth in 3 L bioreactors using DO at 50% air saturation, pH at 7.1, temperature at 37° C. High agitation rate is at 125 rpm and low agitation rate is at 65 rpm. An agitation rate of 125 rpm improved cell growth and cells grew to a higher density.

FIG. 11. Effect of Cytodex™ 1 density on Vero cell growth in 3 L bioreactors. DO was at 50% air saturation, pH was set at 7.1 and temperature was at 37° C. Agitation rate of 125 rpm was used. Two bioreactors contained Cytodex™ 1 at 2 g/L and two at 4 g/L. Cultures with 4 g/L of Cytodex™ 1 had higher cell density than the culture with 2 g/L of Cytodex™ 1.

FIG. 12. Effects of Cytodex™ 1 density on RSV ΔM2-2 Production. Cells were infected with RSV ΔM2-2 virus at MOI of 0.01 and cultured in a shake incubator at 33° C. and 5% CO2 with shaking at 100 rpm. Cultures with 4 g/L of Cytodex™ 1 produced higher virus titer than the culture with 2 g/L of the microcarrier beads.

FIG. 13. Vero cell growth curve in bioreactors which were cultured with 4 g/L of Cytodex™ 1, 125 rpm agitation rate, 50% of DO, pH at 7.1 and 37° C. for 3 days.

FIG. 14. Production of MEDI-559 in a bioreactor. Duplicate bioreactor cultures were inoculated with Vero cells at 2e5 cells/mL in the serum-free growth medium and cultured with 4 g/L of Cytodex™ 1, 125 rpm agitation rate, 50% of DO, pH at 7.1 and 37° C. for 3 days. Cell growth was monitored by taking samples daily from each bioreactor and counting nuclei number in the culture samples. On day 3 of culturing, agitation was stopped to allow the microcarrier beads to settle to the bottom of the bioreactor. Spent growth medium was then removed from the bioreactor while leaving the cells on the microcarrier beads behind and was replaced with equal volume of fresh post-infection medium (SFM4 MegaVir™+4 mM L-Gln). Agitation was resumed at 125 rpm. Temperature of the culture was reduced to 30° C. and pH was set at 7.0. Cells were then infected with MEDI-559 at the MOI of 0.01 and continued to be cultured at 30° C. for 10 days. Samples were taken from the cultures daily from day 7 to day 10. Virus titers were determined by TCID₅₀ assay. Using a straight batch process, a productivity of approximately 8 log₁₀ TCID₅₀/ml was achieved.

FIG. 15. Infectious MEDI-560 titer in the spent culture medium from the following infection conditions: (⋄) SFM4 MegaVir medium and 30° C., (▪) in William's medium E at 30° C., (♦) in SFM4 MegaVir medium at 32° C., (□) in William's medium E at 32° C., and (Δ) in Ex-Cell Vero medium at 32° C.

FIG. 16. Cell growth profiles of the three bioreactor cultures. (♦) 3L260307-R9; (▴) 3L120407-R10; and (Δ) SUB120407. Cell density was measured in cells per milliliter (cells/mL).

FIG. 17. Cell growth profiles of the three bioreactor cultures. (♦) 3L260307-R9; (▴) 3L120407-R10; and (Δ) SUB120407. Cell density was measured in cells per microcarrier.

FIG. 18A-B. Glucose and lactate profiles of the three bioreactor cultures pre-infection. (♦) 3L260307-R9; (▴) 3L120407-R10; and (A) SUB120407.

FIG. 19A-B. Glutamine and ammonium ion profiles of the three bioreactor cultures pre-infection. (♦) 3L260307-R9; (▴) 3L120407-R10; and (Δ) SUB120407.

FIG. 20A-B. Glucose and lactate profiles of the three bioreactor cultures during the infection phase. (♦) 3L260307-R9; (▴) 3L120407-R10; and (Δ) SUB120407.

FIG. 21A-B. Glutamine and ammonium ion profiles of the three bioreactor cultures during virus infection phase. (♦) 3L260307-R9; (▴) 3L120407-R10; and (Δ) SUB120407.

FIG. 22. Cell growth profiles in bioreactors expanded using four different intermittent agitation regimes for bead-to-bead transfer over time after being split at a 1:5 ratio.

FIG. 23. Cell growth profile in 3 L bioreactor seeded with cells from roller bottle (freshly seeded), bioreactor culture after 1× expansion at 1:5 split ratio (1×1:5 transfer) and cultures derived from two consecutive expansion at 1:5 split ratio in bioreactors (2×1:5 transfer).

FIG. 25. Comparative MEDI-560 productions in bioreactor cultures: seeded with cells from a roller bottle (freshly seeded); after 1× expansion at 1:5 split ratio (1×1:5 transfer); and cultures derived from two consecutive expansion at 1:5 split ratio in bioreactors (2×1:5 transfer).

### 5. DESCRIPTION OF THE INVENTION

The present invention relates to a method for propagating viruses. In certain embodiments, conditions for the propagation of virus are optimized in order to produce a robust, scalable and high-yielding cell culture which would be beneficial, e.g., for manufacture of virus vaccine candidates of the invention. In one embodiment, a chemically defined medium is utilized to avoid or reduce substances of animal origin and increase virus production. Critical parameters can be identified, and the production process can be first optimized in small-scale experiments to determine the scalability, robustness, and reproducibility and subsequently adapted to large scale production of virus.

In certain embodiments, the virus that is propagated using the methods of the invention is a negative strand RNA viruses. In certain embodiments, the virus that is propagated is a non-segmented, negative strand RNA viruses, such as a paramyxovirus. The invention specifically provides a method for propagating parainfluenza virus (PIV) and respiratory syncytial virus (RSV) and metapneumovirus (MPV). Even more specifically, the invention provides a method for propagating PIV with human and bovine sequences. In one embodiment, chimeric human/bovine PIV expresses RSV nucleotide sequences. In a specific embodiment, the virus that is propagated is MEDI-534. In another specific embodiment, the virus that is propagated is MEDI-559. In another specific embodiment, the virus that is propagated is one which has a deletion of an open reading frame, such as, for example M2-2 or NS-1. In another specific embodiment, the virus that is propagated is one which is rcp45 hPIV3 or MEDI-560. In certain embodiments, the virus that is propagated using the methods of the invention is an enveloped viruses. In other embodiments the virus that is propagated is a virus that infects and replicates in attached cells. (See also Section 5.5).

Cells are cultured in medium before infection with the virus, i.e., pre-infection. The pre-infection medium may contain serum such as FBS. Subsequently, the cells are infected with the virus and cultured in medium. The post-infection medium may be substantially free of serum. The virus is subsequently harvested.

In particular, the invention provides for the first time a method for propagating a virus by culturing cells in chemically defined lipid concentrate (CDLC)-supplemented medium. In certain embodiments, the CDLC-supplemented medium is substantially free of serum. In certain, more specific embodiments, the medium is serum-free. In an embodiment, a chemically defined medium is utilized to avoid variability and increase virus production. In another embodiment, the CDLC-supplemented medium contains a maximum serum concentration of 0.5% v/v. In certain embodiments, the CDLC comprises one or more of Pluronic F-68, Ethyl Alcohol, Cholesterol, Tween 80, DL-alpha-Tocopherol Acetate, Stearic Acid, Myristic Acid, Oleic Acid, Linoleic Acid, Palmitic Acid, Palmitoleic Acid, Arachidonic Acid, and Linolenic Acid. In a specific embodiment, the CDLC comprises 100,000 mg/L of Pluronic F-68, 100,00 mg/L of Ethyl Alcohol, 220 mg/L of Cholesterol, 2,200 mg/L of Tween 80, 70 mg/L of DL-alpha-Tocopherol Acetate, 10 mg/L of Stearic Acid, 10 mg/L of Myristic Acid, 10 mg/L of Oleic Acid, 10 mg/L of Linoleic Acid, 10 mg/L of Palmitic Acid, 10 mg/L of Palmitoleic Acid, 2 mg/L of Arachidonic Acid, and 10 mg/L of Linolenic Acid. (See also Section 5.1.1).

In a specific embodiment, the CDLC-supplemented medium is substantially free of serum. In certain embodiments, the medium is a serum-free medium, such as OptiPRO™ SFM or VP-SFM™ or SFM4 MegaVir™ (SFM4MV) or Ex-Cell Vero™ or Williams' E medium. In certain embodiments, the cells used are Vero cells.

The present invention provides further optimized conditions for propagating viruses. In one embodiment, cell titer is maximized prior to culturing cells with the virus. In certain embodiments, the cells are cultured in a medium containing serum before infection with a virus or a viral construct of interest and the cells are cultured in a medium without serum after infection with the virus or viral construct. In a specific embodiment, the serum is fetal bovine serum and is present a concentration of 10% of culture volume, 5% of culture volume, 2% of culture volume, or 0.5% of culture volume. In other preferred embodiments, the virus is cultured with cells that have reduced tumorigenicity and are conducive to virus propagation. In certain embodiments, the cells used for virus propagation are Vero cells. In other embodiments, the cell culture used for virus propagation is a perfusion culture. (See also Section 5.1.2).

In certain embodiments, virus titer is increased by modifying the parameters for the post-infection culture conditions. In particular, the virus can be propagated in serum-free media. Serum-free media may be any serum-free media including but not limited to OptiPRO™ SFM (Gibco Cat 12309-019, 2005) and virus-production serum-free medium (VP-SFM) (Gibco Cat 11681-020, 2005) or SFM4 MegaVir™ (Hyclone) or Ex-Cell Vero™ (SAFC Biosciences) or William's E media (Hyclone). (See Section also 5.1.1).

In another embodiment, the present invention relates to a method of optimal virus production by modifying virus titers used to infect cells. The average number of viruses used to infect a cell is referred to as a multiplicity of infection (MOI). In a specific embodiment, MOI used to infect Vero cells is in a range of about 0.0001 to about 0.1. In another embodiment, MOI is in a range of about 0.001 to about 0.01. In one embodiment, MOI is 0.001. In another embodiment, the MOI is 0.01. (See also Section 5.3).

In yet another embodiment, the present invention relates to a method of increased virus production by modifying the parameters of the process and culture conditions. In particular, the invention relates to an improved method of a shifting to a lower post-infection cultivation temperature. In a specific embodiment, the cells are cultured at about 37° C., i.e., at 37° C.±1, before infection with the virus and from about 29° C., i.e., at 29° C.±1 to about 35° C., i.e., at 35° C.±1 after infection with the virus. In one embodiment, the cells are cultured at about 33° C., i.e., at 33° C.±1 after infection with the virus. In another embodiment, the cells are cultured at about 30° C., i.e., at 30° C.±1 after infection with the virus. (See also Section 5.2).

Routine assays may be used to optimize individual parameters for a particular host cell-type and/or virus. Small-scale experiments are conducted to identify and optimize critical process parameters and culture conditions for virus production. Specifically, T-flasks (such as, for example, T-25 or T-75 flasks) are utilized in small-scale experiments to identify and optimize critical process parameters and culture conditions for virus production. Subsequently, the virus production processes are increased to mid-scale production, e.g., using roller bottles or spinner flasks. In one embodiment, spinner flasks use microcarriers for mid-scale production of the virus. (See Section 5.4).

Viruses can be propagated using a microcarrier for cell culture. The advantage to using microcarriers is to increase the surface area for the cells grown in culture, especially for adherent cell lines, such as, for example, Vero cells, in order to improve cell growth. Microcarriers used in connection with the present invention may be any microcarrier including but not limited to Pronactin F, Cytodex™ 1 and Cytodex™ 3. In one embodiment, the microcarrier is Cytodex™ 1. In one embodiment, the amount of microcarrier beads used are range from about 2 g/L to about 20 g/L. In another embodiment, the amount of microcarrier beads used are about 2 g/L, about 4 g/L, or about 5 g/L for batch processes. In another embodiment, the amount of microcarrier beads used are about 20 g/L for fed-batch processes.

Once cultured on microcarrier beads, expansion of the viral cell culture may occur with trypsin. To this end, trypsin is used to detach cultured cells from the microcarrier beads to allow the cells to subsequently adhere to new microcarrier beads added to the culture in order to expand and grow.

Alternatively, expansion of the viral cell culture may occur in the absence of trypsin. A direct bead-to-bead transfer to expand viral cell cultures in bioreactors is used instead. Viral cell culture cells are allowed to directly migrate from the microcarrier beads they are attached to, to adhere to freshly added beads in order to expand and grow. One embodiment of the invention for a direct bead-to-bead transfer involves various intermittent agitation of the viral cell culture for the duration of the culture time. In one embodiment, the intermittent agitation is performed in 1, 2, 3 or more cycles. In one embodiment, each cycle may have a duration of up to 5 hours, up to 8 hours, up to 24 hours or for the entire duration of the culture time. In another embodiment, the intermittent agitation is performed at 125 rpm for 5 minutes and then stopped at 0 rpm for 30 minutes in the first cycle. In another embodiment, the intermittent agitation is performed at 125 rpm for 10 minutes and then stopped at 0 rpm for 50 minutes in the first cycle. In another embodiment, the intermittent agitation is performed at 125 rpm for 1 hour and then stopped at 0 rpm for 1 hour in the second cycle. In yet another embodiment, agitation may be constant at 125 rpm in the second cycle. In yet another embodiment, the agitation may be constant at 125 rpm in the third cycle. See Table VIII.

In addition to the above, direct bead-to-bead transfer can also involve expansion of the viral cell culture by splitting the culture by a ratio of 1:1 or 1:5 into fresh growth medium containing microcarrier beads. In one embodiment, the culture is split when the viral cell culture density is ≧1e6 cells/mL.

The viral constructs and methods of the present invention can be used for commercial production of viruses, e.g., for vaccine production. For commercial production of a vaccine, it is preferred that the vaccine contains only the live attenuated viruses that have been propagated. Contamination of vaccines with adventitious agents introduced during production should also be avoided. Methods known in the art for large scale production of viruses or viral proteins can be used for commercial production of a vaccine of the invention.

In one embodiment, for commercial production of a vaccine of the invention, cells are cultured in a bioreactor or fermenter. Bioreactors are available in volumes from under 1 liter to in excess of 100 liters, e.g., Cyto3 Bioreactor (Osmonics, Minnetonka, Minn.); NBS bioreactors (New Brunswick Scientific, Edison, N.J.); and laboratory and commercial scale bioreactors from B. Braun Biotech International (B. Braun Biotech, Melsungen, Germany). In one embodiment, the viral cell cultures of the invention are grown in a 3 L bioreactor. In one embodiment, the viral cell cultures of the invention are grown in a 15 L bioreactor. In one embodiment, the viral cell cultures of the invention are grown in a 30 L bioreactor. Such bioreactors can be, for example, a stirred-tank Applikon bioreactor. In a specific embodiment, the viral cell cultures of the invention are grown in suspension culture in a single use bioreactor (SUB). In another embodiment, the viral cell cultures of the invention are grown on microcarrier beads in a SUB. In another embodiment, small-scale process optimization studies are performed before the commercial production of the virus, and the optimized conditions are selected and used for the commercial production of the virus.

In one embodiment, a virus is propagated as follows: cells in which the virus is known to grow well are grown under their optimal growth conditions, e.g., with serum and at 37° C.; cells are placed into the CDLC enriched medium, e.g., medium without serum or substantially free of serum, and the cells are infected with the virus; the infected cells are cultured at a lower temperature than the pre-infection culture, e.g., at 33° C. or 30° C.

Viral cell cultures grown according to the invention may achieve a virus titer obtained of at least 7 log₁₀ TCID₅₀/mL. In another embodiment, the virus titer achieved is at least 7.5 log₁₀ TCID₅₀/mL. In another embodiment, the virus titer achieved is at least 8 log₁₀ TCID₅₀/mL. In another embodiment, the virus titer achieved is at least 8.5 log₁₀ TCID₅₀/mL. In another embodiment, the virus titer achieved is at least 9 log₁₀ TCID₅₀/mL.

Viral cell cultures grown according to the invention may yield a certain number of vaccine doses per virus harvest batch. In one embodiment, the viral cell cultures grown according to the invention may yield at least 1 million, at least 2 million, at least 5 million, at least 9 million, at least 10 million, at least 11 million, at least 12 million, at least 15 million, at least 20 million, at least 25 million, at least 30 million, at least 35 million, at least 40 million, at least 45 million, at least 50 million, at least 55 million, at least 60 million, at least 65 million, at least 70 million, at least 75 million, at least 80 million, at least 85 million, at least 90 million, at least 100 million, at least 105 million, at least 110 million, at least 115 million, at least 120 million, at least 125 million, at least 130 million, or at least 135 million vaccine doses per 30 L of virus harvest batch.

### 5.1 Serum-Free Medium

Viral, bacterial and fungal contamination of serum is a concern with regard to the manufacture of biopharmaceuticals. In particular, there have been growing concerns about human exposure to infectious agents such as transmissible spongiform encephalopathies (Asher, 1999, Dev Biol Stand 100: 103-118; Galbraith, 2002, Cytotechnology 39: 117-124) and adventitious viruses (Erickson et al., 1989, Dev Biol Stand 70: 59-66). This has been a driving force behind the adoption of serum-free, animal-free and protein-free media formulations in the manufacturing process (Castle and Robertson, 1999, Dev Biol Stand 99: 191-196). Therefore, serum-free media, or substantially serum-free media, are an excellent alternative to standard serum-containing media for the cultivation of cells.

In certain embodiments, the cells used for viral propagation are cells that can be grown and/or maintained without the addition of components derived from animals or humans. In certain embodiments, the cells used for viral propagation are grown in substantially serum-free medium. In certain embodiments, the cells for virus propagation are cells that are adapted to growth without serum. In a specific embodiment, Vero cells are used for virus propagation. For the purpose of the instant invention, serum-free media may be any serum-free media including but not limited to OptiPRO™ SFM (Gibco Cat 12309-019) and virus-production serum-free medium (VP SFM) (Gibco Cat 11681-020). In certain embodiments, the medium used for culturing of cells infected with the virus that is to be propagated is substantially free of serum.

OptiPRO™ SFM is a serum-free, ultra-low protein (7.5 μg/ml) medium containing no proteins, peptides, or other components of animal or human origin. Stock solutions of the OptiPRO™ SFM are stored at the temperature between 2° C. to 8° C. in the dark. OptiPRO™ SFM is unique in that it supports the growth of numerous attachment-dependent cell lines without the need for addition of attachment proteins to the medium or pre-treatment of the attached surface by inducing the cells to manufacture their own attachment proteins.

VP-SFM is a serum-free, ultra-low protein (5 μg/ml) medium containing no proteins, peptides, or other components of animal or human origin. VP-SFM is available in a ready to use liquid format and is stored at the temperature between 2° C. to 8° C. in the dark.

EX-CELL™ Vero (SAFC BioSciences, JRH Catalog No. 14585) is serum-free and free of animal-derived components. The medium contains a plant-derived hydrolysate and low levels of recombinant proteins, but does not contain phenol red or Pluronic® F68.

In certain embodiments, the serum-free medium is chemically defined, such as FNC Coating Mix® (Athena Environmental Sciences), UltaMEM™ (Cambrex Corporation), HL-1™ (Cambrex Corporation), Neurobasal™ A Medium (Invitrogen), MAM-PF-1,-2-3 (Promocell), RenCyte™ BHK (Medicult), Williams' Medium E (Sigma-Aldrich), and Nutridoma-NS Supplement (Roche). In other embodiments, the serum-free medium is chemically undefined, such as OptiPRO™ SFM and VP-SFM or SFM4 MegaVir™ (Hyclone).

In one embodiment, the present invention relates to a method of increased virus production using a medium that is substantially serum-free. Substantially serum-free medium can be medium that contains less than 5% v/v of serum, less than 2.5% v/v of serum, less than 1% v/v of serum, less than 0.1% v/v of serum, less than 0.01% v/v of serum, or less than 0.001% v/v of serum. In certain embodiments, virus is propagated by incubating cells that are infected with the virus in a culture medium containing less than 5% v/v of serum, less than 2.5% v/v of serum, less than 1% v/v of serum, less than 0.1% v/v of serum, less than 0.01% v/v of serum, or less than 0.001% v/v of serum. In certain embodiments, virus is propagated by incubating cells that are infected with the virus in the complete absence of serum. In certain embodiments, virus is propagated in a serum-free medium comprising chemically defined lipid concentrate (CDLC).

In certain embodiments, the cells are first cultured in medium containing serum and then transferred into medium without serum, by removing the serum-containing medium from the cells and adding the medium without serum. In certain embodiments, the cells are washed with medium without serum to ensure that cells once infected with the virus are incubated in the absence of serum. In certain, more specific embodiments, the cells are washed with medium without serum at least one time, two times, three times, four times, five times, or at least ten times. In certain embodiments, once the cells are in serum-free medium, they are infected with the virus. CDLC can be added before or after the infection with the virus.

5.1.1 Supplementation of Medium with Chemically Defined Lipid Concentrates

The present invention provides for the first time a method for propagating a virus by culturing cells in a medium comprising chemically defined lipid concentrate (CDLC). The present invention contemplates culturing cells in a medium comprising chemically defined lipid concentrate (CDLC) prior to infection with virus. One advantage of using defined medium that is substantially free of serum is the avoidance of risks of contamination and immunogenic stimuli. Contaminants include, but are not limited to, viruses, prions, and mycoplasma. Without being bound by theory, the addition of CDLC to serum-free medium is effective in facilitating cell attachment thereby increasing titers of enveloped viruses in mammalian culture systems.

In one embodiment, the CDLC-supplemented medium is substantially free of serum. In another embodiment, the CDLC-supplemented medium contains a maximum serum concentration of 0.5% v/v. In certain embodiments, the CDLC-supplemented medium contains one or more of Pluronic F-68, Ethyl Alcohol, Cholesterol, Tween 80, DL-alpha-Tocopherol Acetate, Stearic Acid, Myristic Acid, Oleic Acid, Linoleic Acid, Palmitic Acid, Palmitoleic Acid, Arachidonic Acid, and Linolenic Acid. In other embodiments, the CDLC comprises 100,000 mg/L of Pluronic F-68, 100,00 mg/L of Ethyl Alcohol, 220 mg/L of Cholesterol, 2,200 mg/L of Tween 80, 70 mg/L of DL-alpha-Tocopherol Acetate, 10 mg/L of Stearic Acid, 10 mg/L of Myristic Acid, 10 mg/L of Oleic Acid, 10 mg/L of Linoleic Acid, 10 mg/L of Palmitic Acid, 10 mg/L of Palmitoleic Acid, 2 mg/L of Arachidonic Acid, and 10 mg/L of Linolenic Acid. In other embodiments, the CDLC comprises 50,000 to 250,000 mg/L of Pluronic F-68, 50,000 to 250,000 mg/L of Ethyl Alcohol, 100 to 300 mg/L of Cholesterol, 1,000 to 4,000 mg/L of Tween 80, 50 to 100 mg/L of DL-alpha-Tocopherol Acetate, 5 to 20 mg/L of Stearic Acid, 5 to 20 mg/L of Myristic Acid, 5 to 20 mg/L of Oleic Acid, 5 to 20 mg/L of Linoleic Acid, 5 to 20 mg/L of Palmitic Acid, 5 to 20 mg/L of Palmitoleic Acid, 1 to 5 mg/L of Arachidonic Acid, and 5 to 20 mg/L of Linolenic Acid.

In certain embodiments, the CDLC-supplemented medium contains 0.1% v/v to 5% v/v CDLC. In certain embodiments, the CDLC-supplemented medium contains 0.1% v/v, 0.5% v/v, 1% v/v, 2% v/v, 3% v/v, 4% v/v, or 5% v/v. In another embodiment, the CDLC-supplemented medium contains 1% v/v of CDLC.

In certain embodiments, the CDLC is added to the cell culture medium pre-infection. In other embodiments, at least one lipid is exogenously added to the post-infection medium.

5.1.2 Serum Supplementation of Pre-Infection Medium

In certain embodiments, the cells are cultured in a medium containing serum before infection with a virus or a viral construct of interest and the cells are cultured in a medium without serum after infection with the virus or viral construct. In a specific embodiment, the serum is fetal bovine serum and is present a concentration of 10% of culture volume, 5% of culture volume, 2% of culture volume, or 0.5% of culture volume. In certain embodiments, the serum can be, but is not limited to, bovine calf serum, human serum, newborn bovine serum, newborn calf serum, donor bovine serum, donor horse serum.

In certain embodiments, the cells are incubated before infection with the virus in medium containing serum. In certain embodiments, subsequent to infection of the cells with the virus, the cells are incubated in the absence of serum. In other embodiments, the cells are first incubated in medium containing serum; the cells are then transferred into medium without serum; and subsequently, the cells are infected with the virus and further incubated in the absence of virus.

In certain embodiments, the cells are transferred from medium containing serum into medium in the absence of serum, by removing the serum-containing medium from the cells and adding the medium without serum. In other embodiments, the cells are centrifuged and the medium containing serum is removed and medium without serum is added. In certain embodiments, the cells are washed with medium without serum to ensure that cells once infected with the virus are incubated in the absence of serum. In certain, more specific embodiments, the cells are washed with medium without serum at least one time, two times, three times, four times, five times, or at least ten times.

### 5.2 Temperature Shift from Pre-Infection to Post-Infection

In certain embodiments, cells are cultured in a medium containing serum and at a temperature that is optimal for the growth of the cells before infection with a virus or transfection with a viral construct of interest, and the cell culture is incubated at a lower temperature (relative to the standard incubation temperature for the corresponding virus or viral vector) after infection with the virus or transfection with the viral construct of interest. In a specific embodiment, cells are cultured in a medium containing serum before infection with a virus or transfection with a viral construct of interest at 37° C., or about 37° C. (i.e., 37±1° C.) and the cell culture is incubated at 33° C. or about 33° C. (i.e., 33±1° C.) after infection with the virus or transfection with the viral construct of interest. In another embodiment, cells are cultured in a medium containing serum before infection with a virus or transfection with a viral construct of interest at 37° C., or about 37° C. (i.e., 37±1° C.) and the cell culture is incubated at 30° C. or about 30° C. (i.e., 30±1° C.) after infection with the virus or transfection with the viral construct of interest.

In even other embodiments, cells are cultured in a medium containing serum and at a temperature that is optimal for the growth of the cells before infection with a virus or transfection with the viral construct of interest, and the cell culture is incubated without serum at a lower temperature (relative to the standard incubation temperature for the corresponding virus or viral vector) after infection with the virus or transfection with the viral construct of interest. In a specific embodiment, cells are cultured in a medium containing serum before infection with a virus or transfection with a viral construct of interest at 37° C., and the cell culture is incubated without serum at 33° C. or about 33° C. (e.g., 33±1° C.) after infection with the virus or transfection with the viral construct of interest. In another embodiment, cells are cultured in a medium containing serum before infection with a virus or transfection with a viral construct of interest at 37° C., and the cell culture is incubated without serum at 30° C. or about 30° C. (e.g., 30±1° C.) after infection with the virus or transfection with the viral construct of interest.

In certain embodiments, a cell culture infected with a virus or transfected with a viral construct of interest and is incubated at a lower post-infection incubation temperature as compared to the standard incubation temperature for the cells in culture. In a specific embodiment, a cell culture infected with a virus or transfected with a viral construct of interest is incubated at 33° C. or about 33° C. (e.g., 33±1° C.). In certain embodiments, the post-infection incubation temperature is about 25° C., 26° C., 27° C., 28° C., 29° C., 30° C., 31° C., 32° C., 33° C., 34° C., 35° C., 36° C. or 37° C.

In certain embodiments, virus is propagated by incubating a cells before infection with the virus at a temperature optimized for the growth of the cells and subsequent to infection of the cells with the virus, i.e., post-infection, the temperature is shifted to a lower temperature. In certain embodiments the shift is at least 1° C., 2° C., 3° C., 4° C., 5° C., 6° C., 7° C., 8° C., 9° C., 10° C., 11° C., or at least 12° C. In certain embodiments the shift is at most 1° C., 2° C., 3° C., 4° C., 5° C., 6° C., 7° C., 8° C., 9° C., 10° C., 11° C., or at most 12° C. In a specific embodiment, the shift is 4° C.

### 5.3 Multiplicity of Infection

The present invention relates to a method of optimal virus production by modifying virus titers used to infect cells. The average number of viruses used to infect a single cell is referred to as a multiplicity of infection (MOI). In virus manufacturing, an optimal balance of MOI is preferred. Less virus inoculums per infection can extend the lifespan of master virus banks, but an increased virus inoculums can yield higher virus titer.

In one embodiment, MOI used to infect Vero cells is in a range of about 0.0001 to about 0.1. In another embodiment, MOI is in a range of about 0.001 to about 0.01. In a specific embodiment of the invention, a cell culture is infected with the virus having the MOI of about 0.1. In another specific embodiment, a cell culture is infected with the virus having the MOI of about 0.01. In yet another specific embodiment, a cell culture is infected with the virus having the MOI of about 0.001.

In certain embodiments, a cell culture is infected with a virus having the MOI of about 0.0005, 0.0006, 0.0007, 0.0008, 0.0009, 0.001, 0.0011, 0.0012, 0.0013, 0.0014, or 0.0015. In another embodiment, a cell culture is infected with the virus having the MOI of 0.001.

### 5.4 Small-, Mid-, and Large-Scale Virus Production

In one embodiment, small-scale experiments are conducted to identify and optimize critical process parameters and culture conditions for virus production. In a specific embodiment, T-flasks are utilized in small-scale experiments to identify and optimize critical process parameters and culture conditions for virus production. In another embodiment, T-flask experiments are conducted to investigate the combined effects of time of infection and post-infection cultivation temperature on MEDI-534 or MEDI-559 or other viral construct production as disclosed herein, such as, but not limited to a ΔM2-2, ΔNS-1, or rcp45 hPIV3 (MEDI-560). In yet another embodiment, T-flask experiments can assess the impact of pre-infection culture media on virus production.

In one embodiment of the invention, the virus production processes is increased to mid-scale production. In a specific embodiment, roller bottles are used for mid-scale production of the virus. In another specific embodiment, spinner flasks are used for mid-scale production of the virus. In one embodiment, spinner flasks use microcarriers for mid-scale production of the virus. In another embodiment, roller bottles use microcarriers for mid-scale production of the virus.

In one aspect of the invention, the virus production is performed in microcarrier cultures. Microcarrier culture is a technique which makes possible the practical high yield culture of anchorage-dependent cells. The microcarriers provide convenient surfaces for the growth of animal cells and can be used in suspension culture systems or to increase the yield from monolayer culture vessels and perfusion chambers. Microcarriers' application include production of large quantities of cells, viruses and recombinant cell products, studies on cell adhesion, differentiation and cell function, perfusion column culture systems, harvesting cells, etc. In certain embodiments, the microcarriers used are Cytodex™ 1 and Cytodex™ 3 (Amersham Biosciences). In other embodiments, the microcarrier used is Pronectin® F (Sayno Chemical Industries).

The commonly used microcarriers are Cytodex™ 1 and Cytodex™ 3 (Amersham Biosciences). They were specifically developed for the culture of a wide range of animal cells in culture volumes ranging from a few milliliters to thousand liters. Using Cytodex™ in simple suspension culture systems provides yields of several million cells per milliliter.

Cytodex™ is designed to meet the special requirements of a microcarrier technique, which are: the size and density are optimized to give good growth and high yields for a wide variety of cells; the matrix is biologically inert and provides a strong but non-rigid substrate for stirred microcarrier cultures; the microcarriers are transparent allowing easy microscopic examination of the attached cells.

Cytodex™ 1 is based on a cross-linked dextran matrix which is substituted with positively charged N,N-diethylaminoethyl groups. The charged groups are distributed throughout the microcarrier matrix. Cytodex™ 1 is suitable for general purpose microcarrier culture, particularly for most established cell lines. This microcarrier can also be used for production from cultures of primary cells and normal diploid cell strains when maximum recovery of culture products is not essential.

Cytodex™ 3 consists of a thin layer of denaturated collagen chemically coupled to a matrix of cross-linked dextran. The denaturated collagen layer on Cytodex™ 3 is susceptible to digestion by a variety of proteases, including trypsin and collagenase, and provides unique opportunities for removing cells from microcarriers while maintaining maximum cell viability, function and integrity. Cytodex™ 3 is the microcarrier of choice for cells known to be difficult to grow in culture, for differentiated cell culture systems and particularly for cells with an epithelial-like morphology. It can be used as a general purpose collagen-coated culture surface.

In one aspect of the invention, the virus production is performed in microcarrier cultures. In one embodiment, Vero cells are cultured in OptiPRO™ SFM supplemented with FBS and containing Cytodex™ 1. In a particular embodiment, a concentration of FBS is about 0.5% (v/v) to about 2.0% (v/v). In another particular embodiment, a concentration of FBS is about 0.5% (v/v) to about 1.0% (v/v). In one embodiment, a concentration of FBS is about 0.5% (v/v). In a specific embodiment, a concentration of Cytodex™ 1 is about 1 g/L to about 5 g/L. In another specific embodiment, a concentration of Cytodex™ 1 is about 1 g/L to about 3 g/L. In another embodiment, a concentration of Cytodex™ 1 is about 2 g/L. In yet another embodiment, a concentration of Cytodex™ 1 is about 4 g/L.

In another embodiment of the invention, Vero cells are cultured in OptiPRO™ SFM supplemented with FBS and containing Cytodex™ 3. In a particular embodiment, a concentration of FBS is about 0.5% (v/v) to about 2.0% (v/v). In another particular embodiment, a concentration of FBS is about 0.5% (v/v) to about 1.0% (v/v). In one embodiment, a concentration of FBS is about 0.5% (v/v). In a specific embodiment, a concentration of Cytodex™ 3 is about 1 g/L to about 5 g/L. In another specific embodiment, a concentration of Cytodex™ 3 is about 1 g/L to about 3 g/L. In another embodiment, a concentration of Cytodex™ 3 is about 2 g/L.

In yet another embodiment of the invention, Vero cells are cultured in VP-SFM supplemented with CDLC and containing Cytodex™ 1. In a particular embodiment, a concentration of CDLC is about 0.5% (v/v) to about 2.0% (v/v). In another particular embodiment, a concentration of CDLC is about 0.5% (v/v) to about 1.0% (v/v). In one embodiment, a concentration of CDLC is about 1.0% (v/v). In a specific embodiment, a concentration of Cytodex™ 1 is about 1 g/L to about 5 g/L. In another specific embodiment, a concentration of Cytodex™ 1 is about 1 g/L to about 3 g/L. In another embodiment, a concentration of Cytodex™ 1 is about 2 g/L. In yet another embodiment, a concentration of Cytodex™ 1 is about 4 g/L.

In another embodiment of the invention, Vero cells are cultured in VP-SFM supplemented with CDLC and containing Cytodex™ 3. In a particular embodiment, a concentration of CDLC is about 0.5% (v/v) to about 2.0% (v/v). In another particular embodiment, a concentration of CDLC is about 0.5% (v/v) to about 1.0% (v/v). In another embodiment, a concentration of CDLC is about 1.0% (v/v). In a specific embodiment, a concentration of Cytodex™ 3 is about 1 g/L to about 5 g/L. In another specific embodiment, a concentration of Cytodex™ 3 is about 1 g/L to about 3 g/L. In another embodiment, a concentration of Cytodex™ 3 is about 2 g/L.

It will be understood by one of skill in the art that during the process of subculturing adherent cells (i.e., proliferating the cells, expanding the cell culture) the cells must be transferred from a confluent support surface (e.g., flask surface, microcarrier, etc) onto a new support surface. A number of methods can be utilized to effect such cell transfer. For example, proteases, including trypsin and collagenase, may be used to remove cells from flasks or microcarriers the cells are then washed and diluted into a larger flask or into a larger volume of microcarrier containing media for expansion. It is preferable to use a non-animal derived protease for such applications such as, TrypLE (Invitrogen, Carlsbad, Calif.).

Alternatively, in microcarrier cultures direct bead to bead transfer methods may be utilized, wherein fresh beads and media are mixed with the confluent beads and the culture is incubated under conditions which facilitate the transfer of cells to the new beads in order to expand and grow the cell culture. Such bead to bead transfer may utilize an intermittent agitation scheme, such as, for example, agitation of the culture containing microcarrier beads at 100-130 rpm for 5 to 10 minute intervals, cease agitation for up to 40 minutes, for up to 50 minutes, for up to 60 minutes. Then, resume this pattern of agitation for a period of 4 hours, for 5 hours, for 6 hours, for 7 hours, for 8 hours up to overnight (12-24 hours). Then, resume a constant agitation rate at 100-130 rpm until a desired viable cell density is attained. This pattern of intermittent agitation may be repeated until a desired viable cell density is attained. This bead to bead transfer step may be employed without TrypLE to assist in the detachment of cells from one bead to another.

Methods known in the art for large-scale production of viruses or viral proteins can be used for commercial production of a vaccine of the invention. In one embodiment, for commercial production of a vaccine of the invention, cells are cultured in a bioreactor or fermenter. Bioreactors are available in volumes from under 1 liter to in excess of 100 liters, e.g., Cyto3 Bioreactor (Osmonics, Minnetonka, Minn.); NBS bioreactors (New Brunswick Scientific, Edison, N.J.); and laboratory and commercial scale bioreactors from B. Braun Biotech International (B. Braun Biotech, Melsungen, Germany). In another embodiment, small-scale process optimization studies are performed before the commercial production of the virus, and the optimized conditions are selected and used for the commercial production of the virus.

In certain embodiments, a reactor system comprising disposable elements such as a flexible plastic bag for culturing cells is utilized. Such reactor systems are known in the art and are available commercially. See for example International Patent Publications WO 05/108546; WO 05/104706; and WO 05/10849 and Section 6.14 infra. Reactor systems comprising disposable elements (also referred herein as “single use bioreactor(s)” or by the abbreviation “SUB(s)”) may be pre-sterilized and do not require a steam-in-place (SIP) or clean-in-place (CIP) environment for changing from batch to batch or product to product in a culture or production system. As such, SUBs require less regulatory control by assuring zero batch-to-batch I contamination and can, thus, be operated at a considerable cost-advantage and with minimal or no preparation prior to use. Additionally, since SUBs do not require cleaning or sterilizing, they can be rapidly deployed to facilitate production of large quantities of vaccine material (e.g., virus) from cell culture. In particular embodiments, a disposable reactor system is a stirred-tank reactor system which allows for a hydrodynamic environment for mixing the cell culture which allows for more efficient nutrient, O₂ and pH control.

The present invention provides methods for the production of virus in a stirred-tank SUB wherein one or more parameters selected from the group consisting of temperature, agitation rate, pH, dissolved oxygen (DO), O₂ and CO₂ flowrate, are monitored and/or controlled. It will be understood by one of skill in the art that prior to production of a virus the host cells (e.g., Vero cells) must be proliferated to an appropriate cell density to facilitate the propagation of the virus. Accordingly, the present invention further provides methods of proliferating cells (e.g., Vero cells of the present invention) in culture to high cell density by culturing said cells in a SUB.

In one embodiment, cultivation of the cells and/or the virus production is performed in a bioreactor (e.g., a SUB) at a CO₂ concentration of at least 1%, or of at least 2%, or of at least 3%, or of at least 4%, or of at least 5%, or of at least 6%, or of at least 7%, or of at least 8%, or of at least 9%, or of at least 10%, or of at least 20%. In certain embodiments, the CO₂ flowrate is maintained at between about 0.1 L/min to about 1 L/min.

In one embodiment the dissolved oxygen (DO) concentration (pO₂ value) is advantageously regulated during the cultivation of the cells and/or the production of virus and is in the range from 5% and 95% (based on the air saturation). In certain embodiments, the DO is maintained between about 10% to about 80%, or between about 20% to about 70%, or at about 50%. In a specific embodiment the dissolved oxygen (DO) concentration (pO₂ value) is at least 10%, or at least 20%, or at least 30%, or at least 50%, or at least 60%. In still another embodiment the acceptable range for the DO is between about 100 to about 35%. In a specific embodiment, the DO is maintained at between about 35% to about 50%, or at about 50%. In another specific embodiment, the DO should not drop below about 35%. It will be understood by one of skill in the art that the initial DO may be 100% and that the DO may be allowed to drop down to a predetermined level (e.g., 50%) where it is maintained. The DO is maintained used any method known in the art, such as, for example, by sparging O₂. In certain embodiments, the O₂ flowrate is maintained at less then about 2.0 L/min.

In another embodiment, the pH of the culture medium used for the cultivation of the cells and/or the production of virus is regulated and is in the range from pH 6.4 to pH 8.0, or in the range from pH 6.8 to pH 7.4. In a specific embodiment, the pH of the culture medium is maintained at about 6.4, or at about 6.6, or at about 6.8, or at about 7.0, or at about 7.1, or at about 7.2, or at about 7.3 or at about 7.4, or at about 7.6, or at about 7.8, or at about 8.0. It will be understood by one of skill in the art that the initial pH may be lower or higher then the desired range and that the pH may be allowed to increase or decrease to the desired level (e.g., 7.1) where it is maintained. The pH is maintained by any method known in the art. For example the pH may be controlled by sparging CO₂ and/or by adding acid (e.g., HCL) or base (e.g., NaOH) as needed. In certain embodiments, the pH is regulated by the addition of NaOH and/or the sparging of CO₂.

In certain embodiments, Vero cells are cultured in a SUB system to a cell density of at least 5×10⁵ cells/mL, a least 7.5×10⁵ cells/mL, at least 1×10⁶ cells/mL, at least 2.5×10⁶ cells/mL, at least 5×10⁶ cells/mL, at least 7.5×10⁶ cells/mL, at least 10×10⁶, at least 15×10⁶ cells/mL, at least 20×10⁶ cells/mL, or at least 25×10⁶ cells/mL prior to infection.

In a specific embodiment, Vero cells are cultured in a SUB in a serum-free medium such as those described supra (see, for e.g., Section 5.1). In certain embodiments, the media is supplemented with additional glucose. In yet another specific embodiment, Vero cells are cultured in a SUB as adherent cells on a microcarrier such as those described supra (see, for e.g., paragraphs [00109]-[00111]). In one embodiment, the microcarrier is used at a concentration of between about 1 to about 4 g/L. In another embodiment, the microcarrier is used at a concentration of between about 2 to about 3 g/L. In certain embodiment, the cells are cultured without the supplementation of any media component. In other embodiments, the cells are cultured with the supplementation of glucose and glutamine. In still other embodiments, the cells are cultured with the supplementation of CDLC.

As the viral cultures grow and expand, it will secrete metabolites into the culture media. Measurements of these metabolites can indicate the viability of the cells, either pre or post-infection. In certain embodiments, the viral cell culture post-infection or cell culture supernatant, has a lactate concentration of about 1.0 to 2.0 g/L, more particularly from about 1.25 to 1.5 g/L. In certain embodiments, the viral cell culture post-infection or cell culture supernatant, has a glutamine concentration of about 2.0 to about 4.0 g/L, more particularly from about 2.0 to about 3.0 g/L. In certain embodiments, the viral cell culture post-infection or cell culture supernatant, has a glucose concentration of about 0.5 to 2.5 g/L, more particularly from about 1.5 to 1.75 g/L. In certain embodiments, the viral cell culture post-infection or cell culture supernatant, has an ammonium ion concentration of about 1.25 to about 2.5 mM, more particularly from about 2.0 to about 2.25 mM.

In other embodiments, the virus is recovered (i.e., harvested) from the viral cell culture 2 to 12 days post-infection. In another embodiment, the virus is recovered from the viral cell culture 3 to 4 days post-infection.

In certain embodiments the SUB is seeded with the Vero cells to be cultured at a seeding density of about 1×10⁴ cells/mL to about 5×10⁵ cells/mL. In a specific embodiment, the seeding density is between about 3×10⁴ cells/mL to about 3×10⁵ cells/mL, or between about 7×10⁴ cells/mL to about 2×10⁵ cells/mL, or between about 8×10⁴ cells/mL to about 2×10⁵ cells/mL, or between about 9×10⁴ cells/mL to about 1×10⁵ cells/mL, or between about 1×10⁵ cells/mL to about 2×10⁵ cells/mL. In another specific embodiment, the seeding density is between about 1×10⁵ cells/mL to about 2×10⁵ cells/mL.

In one embodiment, the agitation rate of the SUB is maintained at between about 50 to 150 rpm. In a specific embodiment the rate of agitation is maintained at between about 80 to about 120 rpm, or between about 90 to about 100 rpm. In another specific embodiment, the rate of agitation is maintained at between about 100 to about 125 rpm. In yet another embodiment, agitation rates may be maintained at one rate during the cell culturing, but then altered to another rate at another point during the cell culturing (i.e., intermittent agitation). Agitation rates are controlled by means well known in the art.

In certain embodiments, a medium exchange may be performed after cultivation of the cells and prior to infection. In one embodiment, the portion of the medium to be exchanged is between about 20% to about 100%, or between about 30% to about 80%, or between about 30% to about 60%, or between about 66% to about 90%. In one embodiment, the medium is exchange with an equal volume of medium. In another embodiment, the medium is exchange with a reduced volume of medium, effectively concentrating the cells. The medium may be exchanged for a medium having the same or different composition. In one embodiment, a growth medium (i.e., a medium used for proliferation of cells) is exchange for an infection medium (i.e., a medium used during infection and viral growth). Non-limiting examples of growth and infection medium are provided in Sections 5.1 and 6). Alternatively, the growth medium is supplemented with and/or comprises additional components (e.g., glucose, trace mineral, amino acids, etc) such that media exchange is not required.

### 5.5 Viruses

In certain embodiments, the virus that is propagated using the methods of the invention is a negative strand RNA virus. In certain embodiment, the virus is a non-segmented, negative strand RNA virus, such as paramyxovirus. In certain embodiments, the viruses that are propagated using the methods of the inventions are parainfluenza virus (PIV) and respiratory syncytial virus (RSV) and metapneumovirus (MPV). In other embodiments, the invention provides a method for propagating PIV with human and bovine sequences. In one embodiment, the virus is a chimeric human/bovine PIV expresses RSV nucleotide sequences. In a specific embodiment, the virus that is propagated is MEDI-534. In another specific embodiment, the virus that is propagated is MEDI-559. In another specific embodiment, the virus that is propagated is one which has a deletion of an open reading frame, such as, for example M2-2 or NS-1. In another specific embodiment, the virus that is propagated is one which is rcp45 hPIV3 or MEDI-560. In certain embodiments, the virus that is propagated using the methods of the invention is an enveloped viruses. In other embodiments the virus that is propagated is a virus that infects and replicates in attached cells.

In a specific embodiment, the virus that is propagated is MEDI-534. MEDI-534 was constructed by first constructing a recombinant bovine PIV3 vector by replacing the fusion (F) and hemagglutinin-neuraminidase (HN) glycoprotein genes in bovine PIV3 with the human PIV3 and NH genes, respectively (Haller et al., 2000, J Virol 74:11626-11635). Subsequently, the RSV F gene was inserted into the bovine-human PIV3 vector backbone to generate a chimeric virus that expresses the RSV F protein (Tang et al., 2003, J Virol 77:10819-10828). The chimeric virus was named MEDI-534 and it functions as a live, attenuated, bivalent vaccine in animal studies: hamsters and non-human primates immunized with MEDI-554 demonstrated protection from challenge with RSV and hPIV3, and the animals produced RSV-neutralizing and hPIV3 hemagglutination-inhibiting serum antibodies (Tang et al., 2003, supra; Tang et al., 2004, supra).

In yet another embodiment, the virus that is propagated is MEDI-559. MEDI-559 is a live, attenuated RSV vaccine, termed rA2 cp248/404/1030ASH that is temperature sensitive, contains point mutations and a gene deletion of the SH gene. It was found to be well-tolerated and safe when administered to infants in Phase I clinical trials (Karron et al., JID vol 191 p. 1093 (2005)) and was able to elicit an immune response in such patients.

In another embodiment, the virus that is propagated is MEDI-560. MEDI-560 is a live, attenuated HPIV3. A derivative called cp45 was produced by 45 cycles of passage of wt HPIV3 at progressively suboptimal temperatures by Dr. Robert Belshe, now at St. Louis University. This biologically-derived vaccine candidate has been evaluated in adults, seropositive and seronegative children and young infants in Phase I and II trials and appears to be satisfactorily attenuated and immunogenic (Karron et al. Pediatr. Inf. Dis. J. 2003, 22: 394-405). The significant point mutations in cp45 were identified by sequence analysis and placed in wild type recombinant HPIV3 individually or in various combinations to assess their associated phenotypes. This identified three major is and attenuating point mutations in the L protein as well as several non-ts attenuating point mutations in the C and F proteins. This virus has now been recovered from cDNA (rcpPIV3), which provides a virus with a known passage history using acceptable substrates.

In certain embodiments, the viruses that are propagated using the methods of the invention are enveloped viruses. Enveloped viruses include, but are not limited to Paramyxovirus, Herpesvirus, Togavirus, Rhabdovirus, Coronavirus. In other embodiments the viruses that are propagated are viruses that infect and replicate in anchorage-dependent cells. Viruses that infect and replicate in anchorage-dependent cells include, but are not limited to Sindbis virus, Vesicular Stomatitis virus, Oncornavirus, Herpes simplex virus, Hepatitis A virus, RSV virus, Parainfluenza virus, Corona virus, FMDV virus, Rabies virus, Polio virus, and Reo virus.

### 5.6 Cells

In certain embodiments, the viruses are propagated using the methods of the invention in a mammalian cell line. In certain embodiments, the viruses are propagated using the methods of the invention in cells that are anchorage dependent. Anchorage-dependent cells used with the methods of the invention can be cell lines derived from anchorage-dependent type cells including, but not limited to human adipose stem cells, human proximal tubule cells, mouse smooth muscle cells, human endothelial cells, human kidney cells, human large intestine cells, dog kidney cells, hamster ovary cell, green monkey kidney cells, rat small intestine cells, human bladder cells, and human prostate cells.

In certain embodiments, the viruses are propagated in kidney-derived cell lines, that include, but are not limited to MDBK cells, MDCK cells, Vero cells, PK-15 cells, and BHK-21 cells. In other embodiments, the viruses are propagated in BHK-21 cells or Vero cells. In another embodiment, the viruses are propagated in Vero cells.

Vero cells, originating from a continuous African green monkey kidney cell line, are the most commonly used cell line for vaccine production and they have demonstrated an absence of tumorigenicity (Vincent-Falquet et al, 1989, Dev Biol Stand 70: 153-156). Human polio and rabies vaccines are currently manufactured commercially in Vero cells (Montagnon, 1989, Dev Biol Stand 93: 119-123), following specified guidelines provided by regulatory authorities on the use of Vero cells for viral vaccine production (WHO, 1987a,b). Vero cells are usually considered to be anchorage-dependent and are typically propagated in static culture or on microcarriers (Yokomizo et al., 2004, Biotechnol Bioeng 85: 506-515; Wu et al., 2004, Vaccine 22: 3858-3864; Berry et al., 1999, Biotechnol Bioeng 62: 12-19), although they can grow in suspension as cell aggregates (Litwin 1992, Cytotechnology 10: 169-1974). Adherent cultures of Vero cells are well-characterized and have an excellent safety record (Montagnon and Vincent-Falquet, 1998, Dev Biol Stand 93: 119-123). Previously, the replication of three viruses closely related to MEDI-534 were compared in different permissive mammalian cell lines (Haller et al., 2003, J Gen Virol 84: 2153-2162). In all instances, Vero cells generated the highest virus titers. Subsequent experiments showed that the MEDI-534 virus maintained the RSV F gene inserts stably for up to 10 serial passages in Vero cells (Tang et al., 2003, J Virol 77: 10819-10828).

In certain embodiments, the cells used for viral propagation are cells that can be grown and/or maintained without the addition of components derived from animals or humans. In certain embodiments, the cells used for viral propagation are cells that can be grown and/or maintained in substantially serum-free medium, or in medium without serum.

In certain embodiments, the cells to be used with the methods of the invention are capable of attachment to fibronectin. Thus, in certain specific embodiments, the cells are grown post-infection while attached to a fibronectin substrate.

### 5.7 Assays

5.7.1 Measurement of Viral Titer

The viral titer can be measured by any method well-known in the art, for example, but not limited to Tissue Culture Infectious Dose at 50% (TCID₅₀) Assay. The assay measures the potency/infectivity of the virus and uses cells that can be infected with the virus, such as Vero cells. In a specific embodiment, the TCID₅₀ Assay is performed as follows: Vero cells are seeded two days prior to the addition of virus-containing samples in a 96 well plate. The cell plate lot number can be recorded and used as a tool for verifying that the cell passage number is greater than or equal to 126 and less than or equal to 148. The plates are 100% confluent; the cells in the plate are distributed in a smooth, continuous monolayer throughout the well. The cell plates are washed using the Skatron™ cell washer. The washed plates are transferred to a 33±1° C., 5±1% CO₂ incubator and incubated for a minimum of 10 minutes. A virus growth media is dispensed into each well of the cell plate before inoculation with the virus. The virus is serially diluted across a 96-well plate containing a Vero cell monolayer and then the inoculated cells are incubated for 6 days. The plates are subsequently washed, fixed with paraformaldehyde, and incubated with Numax® (RSV-F specific MAb). This is followed by incubation with a goat anti-human Horseradish peroxidase conjugate, with color detection using TMB (3,3′,5,5′-Tetramentylbenzidine, Sigma). A spectrophotometer is used to measure the absorbance of the wells, and values are compared against a negative control cutoff value. The units are then used in the Karber equation to determine a log₁₀ TCID₅₀ titer. Samples generated from the same experiment are tested on the same day, and replicates of four are used for each virus sample to minimize inter- and intra-assay variabilities inherent to the TCID₅₀ assay.

Cell yield and cell density can be measured by any method well-known in the art, for example, but not limited using a hemacytometer or the Cedex cell counting and viability testing system (Innovatis Inc., Malvern, Pa.). In one embodiment, cell densities in microcarrier cultures can be determined by counting nuclei released by 0.1% crystal violet in 0.1 M citric acid solution (Hu and Wang, 1987, Biotechnol Bioeng 30: 548-557).

### 5.8 Kits

The present invention also provides kits for carrying out the assay regimens of the invention. In one embodiment, a kits of the invention comprises serum-free medium and lipid concentrate. In a specific embodiment, the serum-free medium in the kit is OptiPRO™ SFM or VP-SFM or SFM4 MegaVir™ and the lipid concentrate is CDLC. In another embodiment, the kit may comprise, in one or more containers, serum-free medium, lipid concentrate, and vials of cells that can be infected with an enveloped virus. In a specific embodiment, the kit contains one or more vials of Vero cells. In another embodiment, the kit comprises serum-free medium, lipid concentrate, one or more vials of cells that can be infected with an enveloped virus, and one or more vials of an enveloped virus. In a specific embodiment, the kit contains one or more vials of MEDI-534 or MEDI-559. In another embodiment, the kits of the invention include a manual for conducting a method of the present invention. In a specific embodiment, the manual describes the propagation of a virus as follows: cells in which the virus is known to grow well are grown under their optimal growth conditions, e.g., with serum and at 37° C.; cells are placed into the CDLC enriched medium, e.g., medium without serum or substantially free of serum, and the cells are infected with the virus; the infected cells are cultured at a lower temperature than the pre-infection culture, e.g., at 33° C. or 30° C.

### 5.9 Embodiments of the Invention

1. A method for propagating a virus in Vero cells comprising:
   a. culturing the Vero cells in a bioreactor at a first temperature, comprising seeding a cell culture medium containing chemically-defined lipid concentrate (CDLC) and microcarriers with the Vero cells;
   b. infecting the Vero cells cultured in step (a) at a second temperature at a multiplicity of infection of about 0.001 to about 0.10, wherein said second temperature is lower than said first temperature; and
   c. recovering the virus from the cell culture of step (c), wherein said recovered virus yields a viral titer of at least 7.0 log₁₀ TCID₅₀/ml.
2. The method of embodiment 1, wherein said bioreactor is a single use bioreactor (SUB) system.
3. The method of embodiment 2, wherein the SUB is a stirred tank reactor system.
4. The method of embodiment 1, wherein the cell culture medium is a serum free medium.
5. The method of embodiment 1, wherein the CDLC is added to a concentration of 1% v/v.
6. The method of embodiment 4, wherein in the cell culture medium is a serum free medium selected from the group consisting of OptiPRO™ SFM, VP-SFM, SFM4 MegaVir™, Ex-Cell Vero™, or WME.
7. The method of embodiment 1, wherein the chemically defined lipid concentrate comprises one or more of Pluronic F-68, Ethyl Alcohol, Cholesterol, Tween 80, DL-alpha-Tocopherol Acetate, Stearic Acid, Myristic Acid, Oleic Acid, Linoleic Acid, Palmitic Acid, Palmitoleic Acid, Arachidonic Acid, and Linolenic Acid.
8. The method of embodiment 1, wherein the chemically defined lipid concentrate comprises Pluronic F-68, Ethyl Alcohol, Cholesterol, Tween 80, DL-alpha-Tocopherol Acetate, Stearic Acid, Myristic Acid, Oleic Acid, Linoleic Acid, Palmitic Acid, Palmitoleic Acid, Arachidonic Acid, and Linolenic Acid.
9. The method of embodiment 1, wherein the chemically defined lipid concentrate comprises 100,000 mg/L of Pluronic F-68, 100,00 mg/L of Ethyl Alcohol, 220 mg/L of Cholesterol, 2,200 mg/L of Tween 80, 70 mg/L of DL-alpha-Tocopherol Acetate, 10 mg/L of Stearic Acid, 10 mg/L of Myristic Acid, 10 mg/L of Oleic Acid, 10 mg/L of Linoleic Acid, 10 mg/L of Palmitic Acid, 10 mg/L of Palmitoleic Acid, 2 mg/L of Arachidonic Acid, and 10 mg/L of Linolenic Acid.
10. The method of embodiment 1, wherein step (a) utilizes agitation of the culture at an agitation rate between about 50 to about 150 rpm.
11. The method of embodiment 10, wherein said agitation is intermittent.
12. The method of embodiment 11, wherein said culture conditions of step (a) utilize a dissolved oxygen (DO) amount of between about 35% to about 100%.
13. The method of embodiment 1, wherein the first temperature is between about 36° C. and about 38° C.
14. The method of embodiment 1, wherein the second temperature is between about 30° C. and about 33° C.
15. The method of embodiment 1, wherein the microcarrier concentration is between about 1 to about 4 g/L.
16. The method of embodiment 1, wherein said cell culture of step (a) is at a pH of between about 6.6 to about 7.6.
17. The method of embodiment 1, wherein after step (a) but prior to step (b) about 50% to about 90% of the cell culture medium is exchanged.
18. The method of embodiment 16, wherein the cell culture medium is exchanged for a cell culture media having the same composition.
19. The method of embodiment 16, wherein the cell culture medium is exchanged for a cell culture media having a different composition.
20. The method of embodiment 1, wherein the multiplicity of infection is about 0.01.
21. The method of embodiment 1, wherein the Vero cells are cultured for 2 to 12 days in step (c).
22. The method of embodiment 1, wherein the virus is a negative strand RNA virus.
23. The method of embodiment 21, wherein the negative strand RNA virus is non-segmented.
24. The method of embodiment 22, wherein the virus is a paramyxovirus.
25. The method of embodiment 23, wherein the paramyxovirus is a recombinant parainfluenza virus or a recombinant respiratory syncytial virus or a recombinant metapneumovirus.
26. The method of embodiment 24, wherein the parainfluenza virus is a bovine parainfluenza virus.
27. The method of embodiment 25, wherein the bovine parainfluenza virus further comprises one or more human parainfluenza virus nucleotide sequences.
28. The method of embodiment 26, wherein the recombinant parainfluenza virus further comprises a respiratory syncytial virus nucleotide sequence.
29. The method of embodiment 1, wherein said recovered virus yields a viral titer of at least 8.0 log₁₀ TCID₅₀/ml.
30. The method of embodiment 1, wherein said recovered virus yields a viral titer of at least 9.0 log₁₀ TCID₅₀/ml.
31. The method of embodiment 1, wherein said Vero cells are seeded at a density of between about 0.5×10⁵ to 2×10⁵ cells/ml.
32. The method of embodiment 1, wherein said Vero cells in step (a) are cultured to a cell density of at least about 8×10⁵ cells/ml.
33. The method of embodiment 1, wherein the volume of said cell culture of step (a) is at least 1.5 L.
34. The method of embodiment 2, wherein the volume of said cell culture of step (a) is at least 30 L.
35. A Vero cell culture supernatant, comprising virus in a cell culture medium substantially free of serum, wherein said supernatant yields a viral titer of at least 7.0 log₁₀ TCID₅₀/ml.
36. The supernatant of embodiment 34, wherein said supernatant comprises a glucose concentration of about 0.5 to about 2.5 g/L.
37. The supernatant of embodiment 34, wherein said supernatant comprises a lactate concentration of about 1.0 to about 2.0 g/L.
38. The supernatant of embodiment 34, wherein said supernatant comprises a glutamine concentration of about 2.0 to about 4.0 g/L.
39. The supernatant of embodiment 34, wherein said supernatant comprises an ammonium ion concentration of about 1.25 to about 2.5 mM.
40. The supernatant of embodiment 34, wherein said supernatant yields a viral titer of at least 8.0 log₁₀ TCID₅₀/ml.
41. The supernatant of embodiment 34, wherein the virus is a negative strand RNA virus.
42. The supernatant of embodiment 40, wherein the negative strand RNA virus is non-segmented.
43. The supernatant of embodiment 41, wherein the virus is a paramyxovirus.
44. The supernatant of embodiment 42, wherein the paramyxovirus is a recombinant parainfluenza virus or a recombinant respiratory syncytial virus or a recombinant metapneumovirus.
45. The supernatant of embodiment 43, wherein the parainfluenza virus is a bovine parainfluenza virus
46. The supernatant of embodiment 44, wherein the bovine parainfluenza virus further comprises one or more human parainfluenza virus nucleotide sequences.
47. The supernatant of embodiment 45, wherein the recombinant parainfluenza virus further comprises a respiratory syncytial virus nucleotide sequence.
48. The method of embodiment 10, wherein no trypsin is added in step (a).
49. The method of embodiment 48, wherein said culture in step (a) is split 1:1 or 1:5 with fresh culture medium containing new microcarriers.
50. The method of embodiment 49, wherein said split is performed at least once or at least twice during the culture prior to step (b) or pre-infection.
51. The method of embodiment 1 or 50, wherein said method produces at least 2 million, at least 9 million, at least 12 million, at least 120 million vaccine doses per 30 L virus harvest batch.

### 6. EXAMPLES

A vial of Vero cells (ATCC CCL-81, passage 121) was thawed in DMEM+5% (v/v) FBS and then passaged four times in the FBS-supplemented medium prior to direct adaptation to serum free growth in OptiPRO™ SFM that is free from animal derived components. The serum-free Vero cells were banked after 10-15 passages in OptiPRO™ SFM. The Vero cells used in the experiments provided below originated from the OptiPRO™ SFM banks

### Cell Line and Culture Maintenance

The anchorage-dependent Vero cells were routinely seeded at 5×10⁴ cells/mL in corresponding culture volumes (35 mL for T-75 flasks, 100 mL—for T-225 flasks and 350 mL—for 850 cm² roller bottles (RBs) and passaged 3-5 days post-seeding (dps). For cultures passaged 4-5 dps, a complete medium exchange was performed on each culture three dps. In preparation for subculturing, the spent media was aspirated, and the cells were rinsed twice with DPBS. To detach the Vero cells from the flasks, the cultures were incubated at 37° C. with a 0.05% solution of trypsin-EDTA (3 mL for T-75 flasks, 6 mL for T-225 flasks and 10 mL for RBs) After the cells had detached, an equal volume of lima bean trypsin inhibitor (Worthington Biochemical Corporation, Lakewood, N.J.) was added to quench trypsin activity. For all uninfected Vero cells, T-flask cultures were maintained in 37° C./5% CO₂/95% Rh incubators and RB cultures were placed on a roller bottle apparatus operated at 0.3 rpm in a 37° C. incubator.

The cells were pre-adapted to the media tested in each experiment for at least one passage before initiating the experiment, and the cultures were discarded when the cell passage number exceeded 165. Glutamine-free culture media were always supplemented with 4 mM L-glutamine before each use. The cell culture reagents and supplies were sourced from GIBCO/Invitrogen (Carlsbad, Calif.) and tissue culture wares were purchased from Corning (Corning, N.Y.), unless specified otherwise.

### Virus Constructs and Seed Stock Preparation

Construction of the MEDI-534 virus has been previously detailed (Tang et al., 2003). To generate the virus seed stock used for infection in all the experiments, MEDI-534 obtained by plasmid rescue (Tang et al., 2003, supra) was added at a multiplicity of infection (MOI) of 0.001 to T-225 flask cultures of Vero cells growing for three days in OptiPRO™ SFM. The culture media were collected four dpi and stabilized with 10% (v/v) sucrose phosphate prior to aliquoting into multiple 1 mL cryovials. The virus seed stocks were stored at −80° C. and thawed only immediately before use.

### T-Flask Experiments

T-75 flasks were seeded with 1.75×10⁶ Vero cells in 35 mL OptiPRO™ SFM (unless specified otherwise) and maintained in a 37° C./5% CO₂/95% Rh incubator. At the time of infection, the spent medium was removed from each flask and the cells were rinsed with 2×10 mL DPBS. One flask in each medium condition was trypsinized and the cells were counted. To infect the cultures, DMEM (unless stated otherwise) containing MEDI-534 at the appropriate MOI was added to the remaining flasks. Post-infection, the T-flasks were maintained in humidified incubators with 5% CO₂ overlay.

### Roller Bottle Experiments

Each 850 cm² roller bottle (RB) was seeded with 1.5×10⁷ Vero cells in the growth medium of choice and maintained at 37° C. with constant rotation at 0.3 rpm. In all instances, the basal growth medium was either OptiPRO™ SFM or virus production serum-free medium (VP-SFM), also a commercially available ADCF SFM from GIBCO/Invitrogen (Carlsbad, Calif.). In some instances, the basal growth medium was supplemented with fetal bovine serum (FBS) sourced from JRH Biosciences, Inc. (Lenexa, Kans.) or with a chemically defined lipid concentrate (CDLC) purchased from GIBCO/Invitrogen. To generate growth curves in OptiPRO™+0.5% FBS and VP-SFM+1% CDLC, duplicate RB cultures in each medium were trypsinized and counted daily. To generate virus production profiles, the spent medium was removed from each RB and the cells were rinsed with 300 mL DPBS immediately before infection (at three dps). At least one flask in each medium condition was trypsinized and the cells were counted to determine the cell yield per flask and to calculate the appropriate amount of virus to add for infection at MOI 0.001. Unless stated otherwise, 500 mL of Williams' Medium E (WME)—a chemically defined ADCF basal medium—containing MEDI-534 was added to each of the remaining flasks at the time of infection. Post-infection, all RBs were incubated at 33° C. with constant rotation at 0.3 rpm.

### Spinner Flask Experiments

The growth medium was either OptiPRO™ supplemented with 0.5% (v/v) FBS or VP-SFM supplemented with 1% (v/v) CDLC. Cytodex™ 1 microcarriers were rehydrated and sterilized according to the manufacturer's recommendations (Amersham Biosciences AB, Uppsala, Sweden). The microcarrier beads were then rinsed once with the appropriate growth medium before use. Two hours pre-seeding, each 250 mL glass spinner flask (Bellco Biotechnology, Inc., Vineland, N.J.) was filled with 200 mL of the chosen growth medium containing 2 g/L Cytodex™ 1 and incubated at 37° C./5% CO₂/95% Rh with 60 rpm agitation. To inoculate the spinner flasks, 2×10⁷ Vero cells were added per vessel. All cultures were incubated pre-infection at 37° C./5% CO₂/95% Rh with agitation maintained at 60 rpm. To generate growth curves, well-mixed samples were taken from the spinner flasks daily for nuclei counting. Prior to infection, a sample was removed from each spinner flask to determine the nuclei count and to calculate the amount of virus to infect with at MOI 0.001. In preparation for infection, agitation was stopped for all the spinner flasks. After the microcarrier beads had settled, 90% of the spent medium was replaced by WME containing MEDI-534 at MOI 0.001. Post-infection, the cultures were incubated at 33° C./5% CO₂/95% Rh with constant agitation at 60 rpm.

### Bioreactor Experiments for MEDI-534

Bioreactor experiments were conducted in 3 L stirred tank bioreactors (Applikon, Foster City, Calif.) with dissolved oxygen (DO) maintained at 50% of air saturation. Each bioreactor was equipped with an ADI 1030 Bio Controller (Applikon) and an ADI 1035 Bio Console (Applikon). Cytodex™ 1 microcarriers were prepared for use following the manufacturer's instructions. Three hours pre-seeding, three bioreactors were each filled with 2 L VP-SFM supplemented with 1% (v/v) CDLC and 2 g/L Cytodex™ 1. The bioreactor contents were warmed to 37° C. with heating blankets and agitated at 60 rpm with single marine impellers. The pH setpoints in the three bioreactors were 7.0, 7.2, and 7.4, respectively. The culture pH was controlled at the designated levels by the CO₂ percentage in the inlet gas and by the addition of 1 N NaOH solution after the CO₂ percentage in the inlet gas was reduced to 0%. To ensure that all the cells used an experiment had the same passage history, all the bioreactors in each experiment were inoculated with cells pooled from multiple RBs. Bioreactor contents were sampled daily for nuclei counting to generate growth curves. To prepare for infection, agitation was stopped in the bioreactors. After the microcarrier beads had settled, 90% of the spent medium was replaced by WME containing MEDI-534 at MOI 0.001. Post-infection, the pH and DO setpoints were not changed, but the temperature setpoint was lowered to 33° C. and agitation was increased to 100 rpm.

### Collection of Infected Samples for Virus Quantification

After sampling the media in infected T-flask and RB cultures, 10% (v/v) sucrose phosphate was added to stabilize the virus samples. After sampling from infected spinner flasks and bioreactors, microcarrier beads in the samples were allowed to settle and the culture supernatants collected were stabilized with 10% (v/v) sucrose phosphate. All sucrose phosphate stabilized virus samples were immediately stored at −80° C. until analyses.

### Analytical Methods

Cells from T-flasks and RBs were enumerated either using a hemacytometer or the Cedex cell counting and viability testing system (Innovatis Inc., Malvern, Pa.) operated according to the manufacturer's directions. Cell densities in microcarrier cultures were determined by counting nuclei released by 0.1% crystal violet in 0.1 M citric acid solution (Hu and Wang, 1987, Biotechnol Bioeng 30: 548-557). Infectious virus titers were measured with an in-house 50% tissue culture infective dose (TCID₅₀) assay and results were quantified in log₁₀ TCID₅₀/mL. To minimize inter- and intra-assay variabilities inherent to the TCID₅₀ assay, samples generated from the same experiment were tested on the same day when possible, and replicates of four were used for each virus sample.

6.1 Virus Production Profiles at Different Multiplicity of Infections

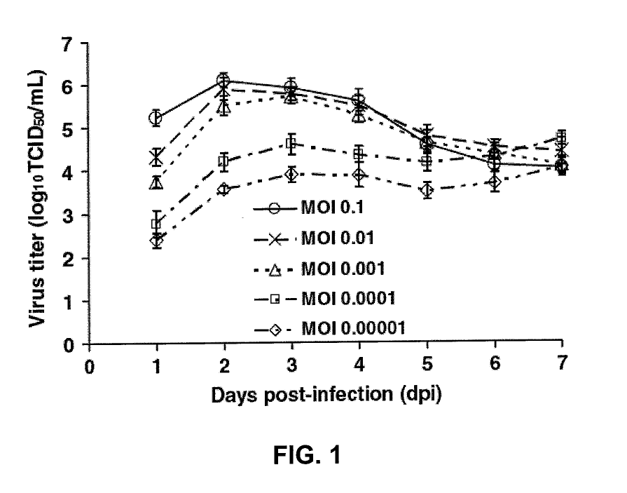

Small-scale T-75 flask experiments were conducted to identify and optimize critical process parameters and culture conditions for MEDI-534 production. The influence of multiplicity of infection (MOI) on MEDI-534 production was examined (FIG. 1). Three MOIs—0.1, 0.01 and 0.001—were shown to generate comparable peak virus titers (6 log₁₀ TCID₅₀/mL), whereas the two lowest MOIs—0.0001 and 0.00001—yielded maximum virus titers that were lower by at least 1 log₁₀ TCID₅₀/mL. Therefore, to conserve virus seed stocks while maximizing virus yields, infection in subsequent experiments employed MOIs ranging from 0.01 to 0.001.

6.2 Effects of Time of Infection and Post-Infection Temperature on Infectious Virus Titers

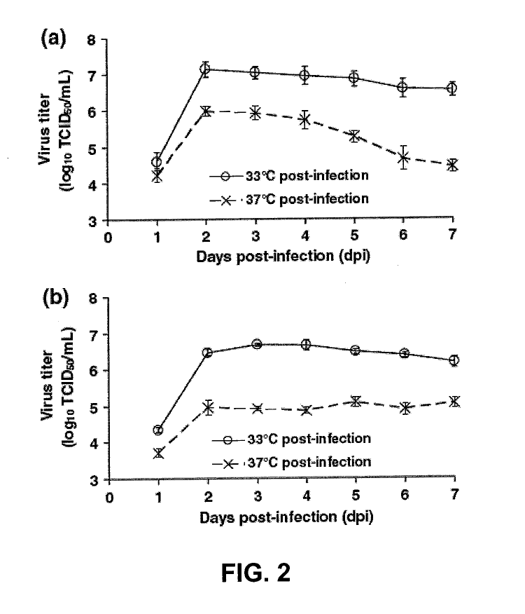

T-flask experiments were conducted to investigate the combined effects of time of infection and post-infection cultivation temperature on MEDI-534 production (FIGS. 2a & 2b). By infecting the cultures at five days post-seeding (dps) in comparison with three dps, the number of Vero cells at infection increased from 0.6×10⁷ cells/flask to 1.7×10⁷ cells/flask. The peak virus titers achieved in cultures infected at three dps (FIG. 2a) were slightly higher that that measured in cultures infected at five dps (FIG. 2b). Therefore, increasing in the Vero cell number at infection did not enhance MEDI-534 production. However, the shift to a lower post-infection cultivation temperature (from 37° C. to 33° C.) markedly elevated peak virus titers by at least 1 log₁₀ TCID₅₀/mL. Follow-up studies showed that the post-infection cultivation temperatures of 29° C., 31° C. and 35° C. demonstrated the same tendency but they yielded slightly lower MEDI-534 titers than the post-infection temperature of 33° C. Therefore, in all further experiments the post-infection incubation temperature of 33° C. was employed.

The observed increase in virus titers at lower cultivation temperatures was unexpected because MEDI-534 was not cold-adapted (Tang et al., 2003, J Virol 77: 10819-10828). Although the specific reasons for this temperature sensitivity are not identified, amino acid changes in viral polymerases have produced PIV3 viruses that exhibit temperature-sensitive phenotypes (Feller et al., 2000, Virology 275: 190-201; Skiadopoulos et al., 1999, J Virol 73: 1374-1381).

6.3 Effect of Pre-Infection Culture Media on Virus Production

A T-flask experiment assessed the impact of pre-infection culture media on virus production (Table I). To enhance the pre-infection growth media, Vero cultures were supplemented with FBS at the time of seeding. Addition of FBS to OptiPRO™ SFM almost doubled the cell yields measured at three dps, and increased the maximum virus titers at least fivefold. FBS supplementation at the 0.5%, 2% and 5% levels produced comparable cell yields (1.0−1.1)×10⁷ cells/flask) and virus titers ((7.6-7.8) log₁₀ TCID₅₀/mL), suggesting that increasing the FBS concentration will not further improve cell growth or virus production. In a similar experiment conducted in a retrovirus packaging cell line, Lee and coworkers doubled retroviral vector titers upon serum addition but they found that virus production was dose-independent in the serum supplementation range tested (1% to 20%) (Lee et al., 1996, Appl Microbiol Biotechnol 45: 477-48).

TABLE I
Comparison of cell yield and MEDI-534 production
in cultures titrated with FBS pre-infection

| Pre-infection culture | Cell Yield at infection | Maximum virus titer |
|---|---|---|
| medium | (cells/flask) | (log₁₀ TCID₅₀/mL |
| DMEM + 5% FBS | 1.1 × 10⁷ | 7.8 ± 0.2 |
| OptiPRO ™ + 2% FBS | 1.0 × 10⁷ | 7.6 ± 0.2 |
| OptiPRO ™ + 0.5% FBS | 1.1 × 10⁷ | 7.7 ± 0.2 |
| OptiPRO ™ SFM | 0.6 × 10⁷ | 6.9 ± 0. |

6.4 Virus Production Profiles of Roller Bottle Cultures Titrated with FBS Pre-Infection Media and Supplements

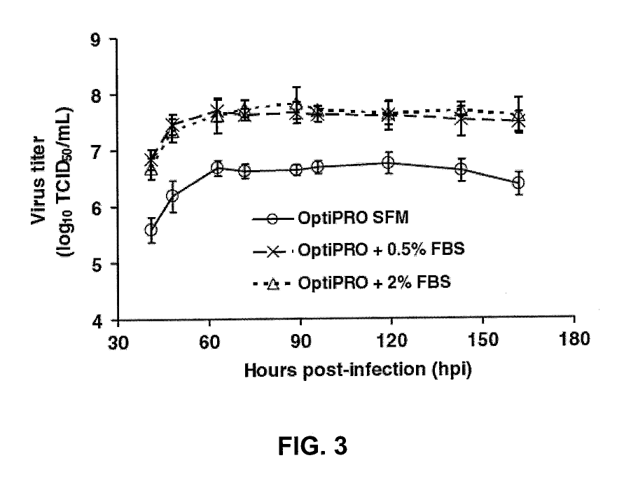

Cell yields and MEDI-534 production were measured in RB cultures supplemented with 0.5% and 2% FBS at the time of seeding to determine the reproducibility and scalability of the FBS titration experiment with T-flasks (FIG. 3). The cell yield for the OptiPRO™ SFM culture was only 1.9×10⁷ cells/RB at three dps, even though the cultures were inoculated at 1.5×10⁷ cells/RB. In contrast, T-flasks cultures of Vero cells, seeded at 1.75×10⁶ cells/flask in OptiPRO™ SFM, tripled in cell numbers after three days in culture (Table I). These results indicate that OptiPRO™ SFM preferentially supports cell growth in static cultures. When OptiPRO™ SFM was supplemented with 0.5% (v/v) and 2% (v/v) FBS, cell growth in the RB cultures correlated with that observed in the T-flask cultures—cell numbers increased about sixfold after three days in both the T-flask (Table I) and the RB cultures (figure legend of FIG. 3). Three dps, the OptiPRO™+0.5% (v/v) FBS and OptiPRO™+2% (v/v) FBS cultures gave comparable cell yields at 9.3×10⁷ cells/RB and 10.4×10⁷ cells/RB, respectively. In accord with the T-flask findings (Table I), the FBS supplemented RB cultures yielded significantly higher virus titers than the OptiPRO™ SFM cultures. The success achieved in supplementing the pre-infection growth medium (OptiPRO™ SFM) with FBS illustrates the impact exerted by the culture medium on cell growth and virus production.

6.5 Cell Yield and Virus Production in Chemically Defined Lipid Concentrate Supplemented Pre-Infection Media

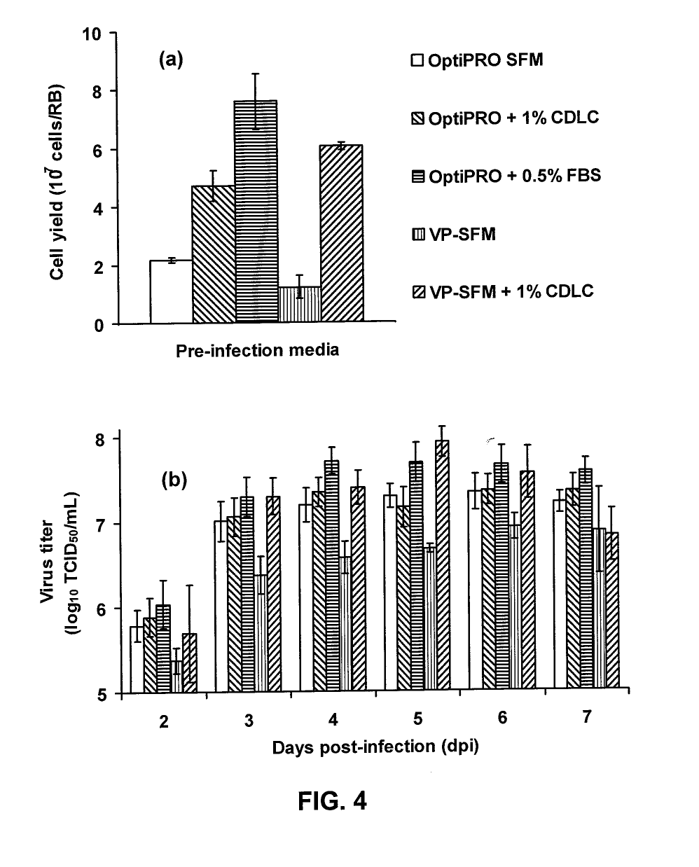

Chemically defined lipid concentrate (CDLC) as a serum-free replacement for FBS was evaluated. In addition, this experiment also compared OptiPRO™ SFM with VP-SFM, another animal derived component free (ADCF) SFM that was also developed by the same manufacturer (GIBCO/Invitrogen) to support Vero growth and vaccine production. Duplicate RBs in the five distinct pre-infection media generated the following cell yields at three dps (FIG. 4a) and peak virus titers (FIG. 4b). Data is presented as mean±standard deviation in Table II.

At three dps, the cell densities barely increased beyond the seeding density of 1.5×10⁷ cells/RB in both the OptiPRO™ SFM and VP-SFM conditions (FIG. 4a). In contrast, cell numbers in the lipid or serum supplemented cultures increased several fold after three days (FIG. 4a). In spite of the superior cell growth in the OptiPRO™+0.5% (v/v) FBS cultures, virus titers were marginally higher in the VP-SFM+1% (v/v) CDLC cultures. The CDLC-associated boost in cell growth and virus production was more marked in the VP-SFM cultures than in the OptiPRO™ SFM cultures. The more pronounced effect of CDLC addition in one SFM over another may have resulted from differences in the lipid composition of the two SFM, but this hypothesis cannot be validated because the composition of both OptiPRO™ SFM and VP-SFM are proprietary to the manufacturer (GIBCO/Invitrogen). This is the first demonstration of successful use of lipid supplementation to enhance cell growth and virus production in Vero cultures.

OptiPRO™ SFM is a chemically undefined SFM that has exhibited discernible lot-to-lot variability in supporting Vero cell growth and MEDI-534 production. Since the first and second RB experiments used different lots of OptiPRO™ SFM, the higher peak virus titers achieved with OptiPRO™ SFM in the second RB experiment ((7.3±0.2) log₁₀ TCID₅₀/mL) versus the first experiment (FIG. 3; (6.8±0.2) log₁₀ TCID₅₀/mL) may have resulted from the lot-to-lot variability in OptiPRO™ SFM, the inter-assay variability in the TCID₅₀ assay, or both.

TABLE II
Comparison of cell yield and MEDI-534 production
in five distinct pre-infection media

| Pre-infection culture | Cell Yield at infection | Maximum virus titer |
|---|---|---|
| medium | (cells/RB) | (log₁₀ TCID₅₀/mL) |
| OptiPRO ™ SFM | (2.2 ± 0.1) × 10⁷ | 7.3 ± 0.2 |
| OptiPRO ™ SFM + 1% | (4.7 ± 0.5) × 10⁷ | 7.4 ± 0.2 |
| (v/v) CDLC | | |
| OptiPRO ™ SFM + 0.5% | (7.6 ± 1.0) × 10⁷ | 7.7 ± 0.2 |
| (v/v) FBS | | |
| VP-SFM | (1.2 ± 0.4) × 10⁷ | 6.9 ± 0.2 |
| VP-SFM + 1% | (6.0 ± 0.1) × 10⁷ | 7.9 ± 0.2 |
| (v/v) CDLC | | |

6.6 Cell Growth and Virus Production in Roller Bottles Using Chemically Defined Lipid Concentrate Supplemented Pre-Infection Media

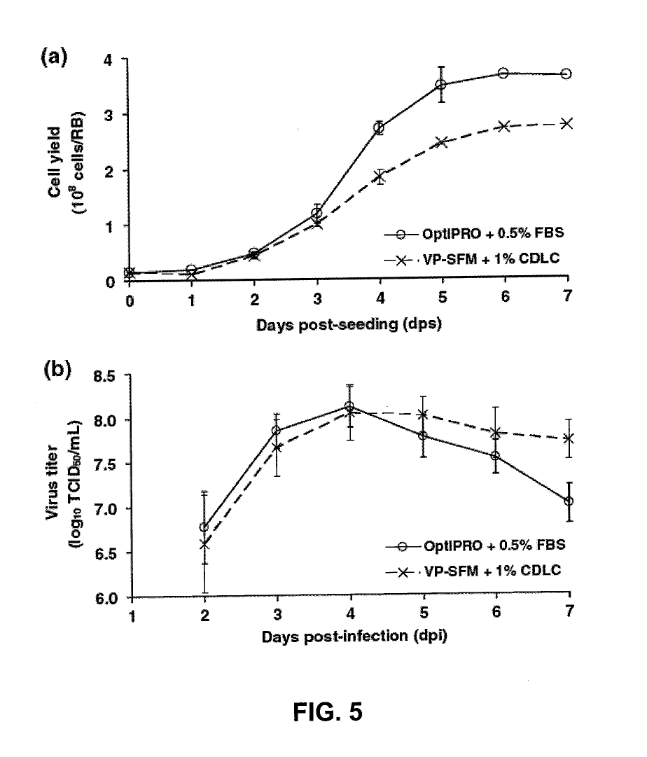

The kinetics of cell growth (FIG. 5a) and virus production (FIG. 5b) in the two pre-infection growth media: OptiPRO™+0.5% (v/v) FBS and VP-SFM+1% (v/v) CDLC were measured. Although the FBS supplemented cultures yielded an approximately 30% more cells than the serum-free cultures, peak virus titers were comparable (8.1 log₁₀ TCID₅₀/mL). The kinetics of MEDI-534 production is apparently influenced by the pre-infection culture media; maximum virus titer was detected at four dpi and five dpi for the OptiPRO™+0.5% (v/v) FBS and VP-SFM 1% (v/v) CDLC cultures, respectively (FIG. 5b).

6.7 Cell Growth and Virus Production in Roller Bottle Cultures Titrated with Chemically Defined Lipid Concentrate Supplemented Pre-Infection Media

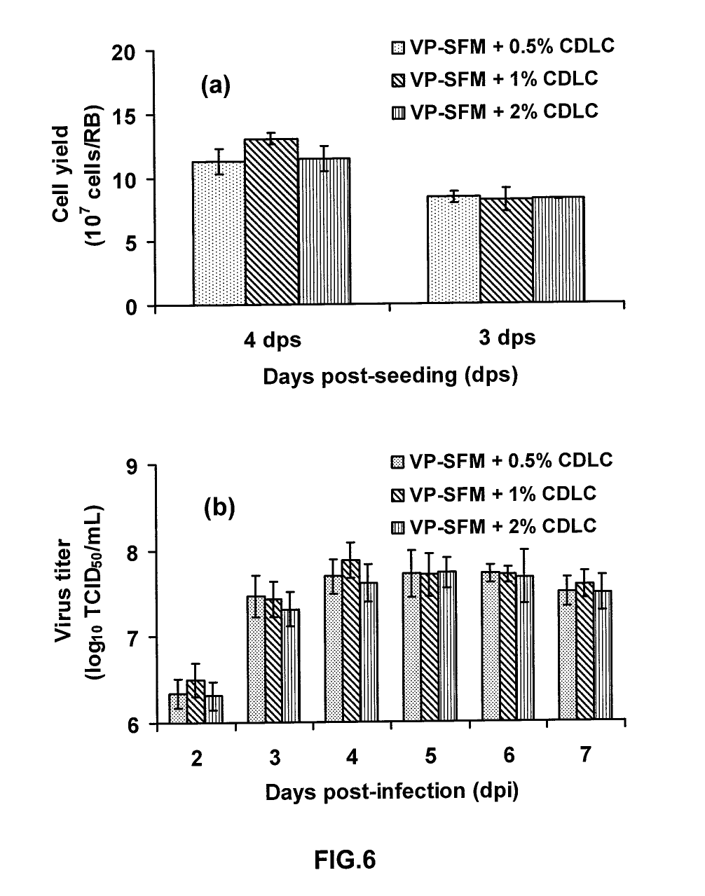

Vero cultures titrated with CDLC at 0.5% (v/v), 1% (v/v) and 2% (v/v) in VP-SFM showed similar cell yields (FIG. 6a) and MEDI-534 production (FIG. 6b). All further experiments employed VP-SFM+1% (v/v) CDLC as the pre-infection serum-free Vero growth medium.

6.8 Medi-534 Production in Chemically Defined Lipid Concentrate Supplemented Post-Infection Media

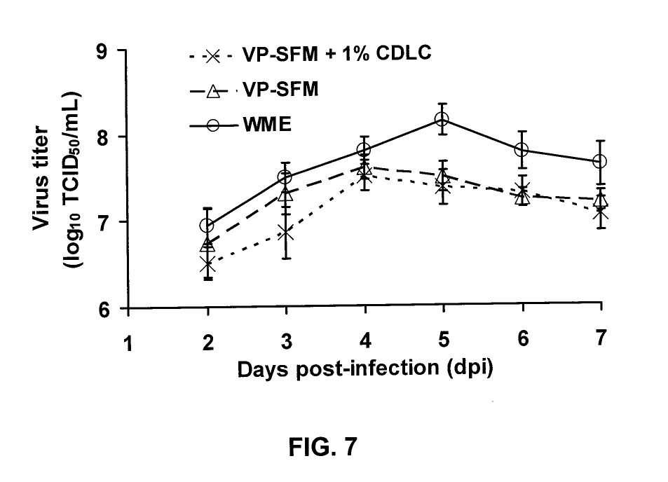

The feasibility of replacing Williams' Medium E (WME), a chemically defined ADCF SFM of known composition (Williams and Gunn, 1974), with VP-SFM or VP-SFM+1% (v/v) CDLC as the post-infection SFM was explored (FIG. 7). Although WME did not support Vero growth in uninfected cultures, it demonstrated superior performance as a post-infection medium. The maximum virus titers (represented as mean±standard deviation) measured in VP-SFM+1% (v/v) CDLC, VP-SFM and WME corresponded to (7.5±0.2) log₁₀ TCID₅₀/mL, (7.6±0.1) log₁₀ TCID₅₀/mL and (8.1±0.2) log₁₀ TCID₅₀/mL, respectively. To minimize the use of animal-derived components in the production process as well as to minimize potential complications in the purification process, serum-supplemented media were not tested. Consequently, ensuing infections with MEDI-534 employed WME exclusively as the virus production medium.

6.9 Cell Growth and Virus Production in Microcarrier Cultures Using Chemically Defined Lipid Concentrate Supplemented Pre-Infection Media

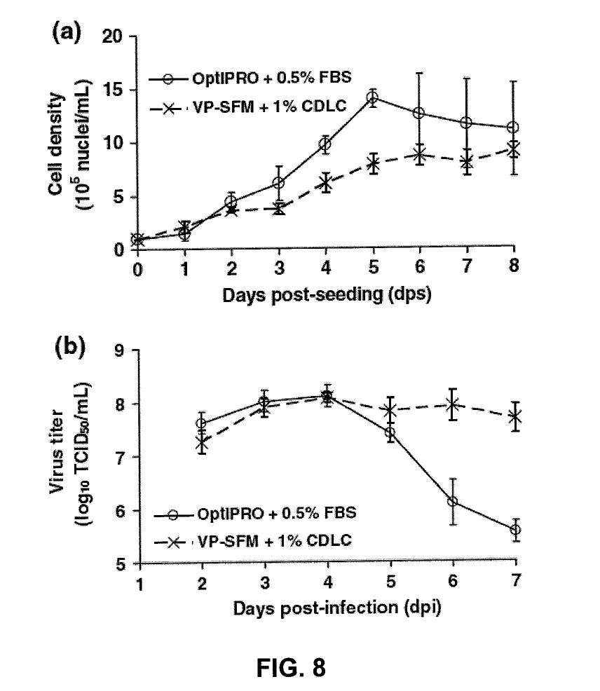

Spinner flask experiments contrasted the abilities of the serum-supplemented (OptiPRO™+0.5% (v/v) FBS) and serum-free (VP-SFM+1% (v/v) CDLC) media to support Vero cell growth (FIG. 8a) and MEDI-534 infection (FIG. 8b) in microcarriers. Vero cells cultured in both media grew on Cytodex™ 1 and Cytodex™ 3. Consistent with the RB observations (FIG. 5a), OptiPRO™+0.5% (v/v) FBS cultures grew faster than VP-SFM+1% (v/v) CDLC cultures. Both pre-infection media yielded identical peak virus titers of 8.1 log₁₀TCID₅₀/mL simultaneously (at four dpi.). However, the infectious virus titer dropped by an average of 2.6 log₁₀ TCID₅₀/mL in the pair of serum-supplemented cultures from four dpi to seven dpi, whereas the virus titer only declined by an average of 0.4 log₁₀ TCID₅₀/mL over the corresponding length of time in the two serum-free cultures. OptiPRO™+0.5% (v/v) cultures in RBs did not exhibit such a substantial loss in infectious virus titer (FIGS. 4b & 5b), and the underlying mechanism for this phenomenon in spinner flask cultures is not known. On the basis of the results in this experiment, all subsequent microcarrier experiments used VP-SFM+1% (v/v) CDLC as the pre-infection growth medium for Vero cells.

6.10 The Effect of Time of Infection on MEDI-534 Titers in Microcarrier Cultures

The effect of time of infection on MEDI-534 titers was considered in microcarrier cultures. In accord with T-flask observations that the virus titers were comparable for cultures infected at 3 dps and 5 dps (Table 1), the duplicate spinner cultures infected on 4,5, and 6 dps yielded nearly equivalent peak MEDI-534 titers (Table II). This “cell density effect” has been observed for adenovirus production in batch cultures: specific virus productivity decreased with increasing cell density at the time of infection (Background of the Invention, Henry et al., 2004, Biotechnol Bioeng 86: 765-774, Nadeau and Kamen, 2003, Biotechnol Adv 20: 475-489). To shorten culture time, microcarrier cultures were infected four dps in the experiments that follow.

In preparation for scale-up in bioreactors, subsequent attempts to optimize microcarrier process parameters—including seeding density, serum-free growth medium, agitation rate, microcarrier type and bead concentration—showed that the current operating conditions (inoculate 1×10⁵ cells/mL in VP-SFM+1% CDLC containing 2 g/L Cytodex™ 1 with 60 rpm agitation) gave the best results.

TABLE III
Virus Production in microcarrier cultures
infected at different times post-seeding

| | Cell density at infection | Maximum virus titer |
|---|---|---|
| Time of infection | (nuclei/mL) | (log₁₀ TCID₅₀/mL) |
| 4 dps | (5.1 ± 0.6) × 10⁵ | 8.2 ± 0.2 |
| 5 dps | (7.4 ± 1.4) × 10⁵ | 8.1 ± 0.2 |
| 6 dps | (9.6 ± 2.5) × 10⁵ | 8.1 ± 0.3 |

6.11 Comparison of Pre-Infection Vero Cell Growth and Virus Production in Bioreactors Controlled at Different pHs.

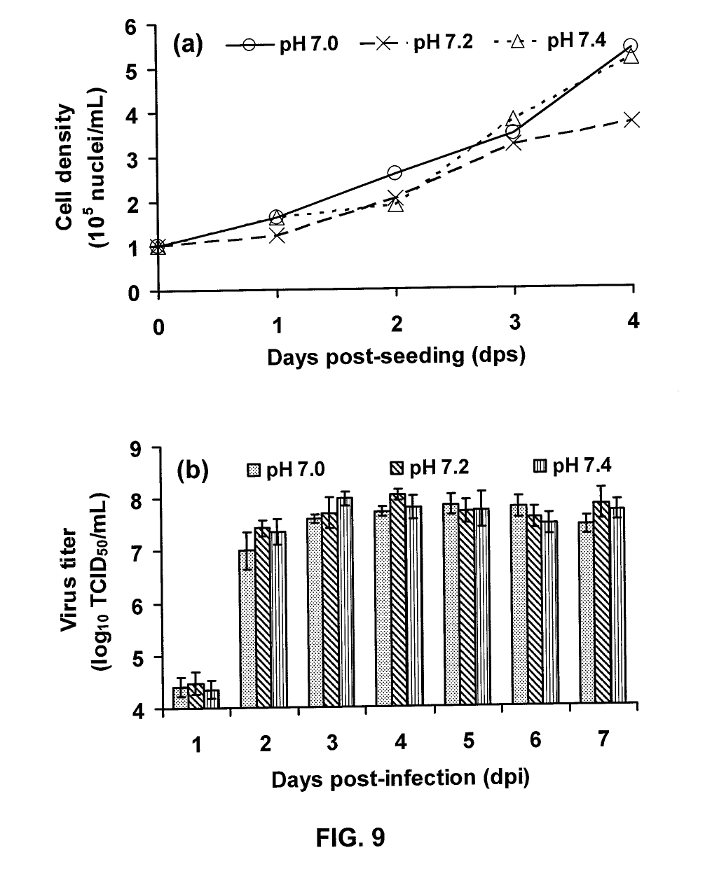

Bioreactor experiments were performed to assess the scalability of the serum-free MEDI-534 production process and evaluate the dependence of cell growth and virus production on culture pH. Three parallel bioreactor cultures maintained pH setpoints at 7.0, 7.2 and 7.4, respectively. Pre-infection cell growth (FIG. 9a) and virus production profiles (FIG. 9b) in the bioreactor cultures were similar to that observed in the VP-SFM+1% CDLC spinner flask cultures (FIG. 8). A repeat of this bioreactor experiment generated the same trends with maximum virus titers of 8 logᵢₙ TCID₅₀/mL. These results demonstrate that the microcarrier process can be scaled up in bioreactors and also show that cell growth and virus production are relatively pH-independent within the pH 7.0-7.4 range. The pH in the T-flask, RB, and spinner flask cultures typically varied considerably—in some instances, pH decreased from 7.6 to 6.8 over the course of culture with no obvious detrimental effects.

6.12 Improving RSV Process Production in Bioreactors

To improve RSV vaccine productivity in a bioreactor process, bettering Vero cell growth was examined using the Applikon 3 L bioreactors. It is likely higher cell density will result in higher virus titer as there are more cells to produce the viruses. In the previous process (see paragraph [00167]), 2 g/L of microcarrier Cytodex™ 1 and 60 rpm agitation rate were used. Higher agitation rates and higher Cytodex™ 1 density on Vero cell growth and RSV vaccine production were examined.

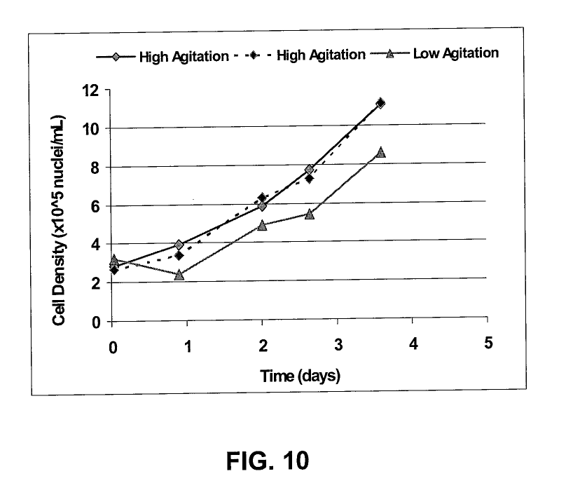

To evaluate the effect of agitation rate on Vero cell growth, four 3 L bioreactors were inoculated with Vero cells at 2e5 cells/mL in 1.5 L culture volume in the Vero growth medium (VP-SFM, 4 mM L-Gln, and 1% CDLC). The following parameters were used for the bioreactor cultures, dissolved oxygen (DO) at 50% air saturation, pH at 7.1, temperature at 37° C. The agitation rate for two of the bioreactors was set at 65 rpm and the other two were set at 125 rpm. Cell growth was monitored by taking samples daily from each bioreactor and counting nuclei using Crystal Blue staining method. The data is shown in FIG. 10. Due to a culture contamination problem, one of the duplicate bioreactor cultures at the 65 rpm was lost. The high agitation rate is at 125 rpm and the low agitation rate is at 65 rpm. As shown in FIG. 10, an agitation rate of 125 rpm improved cell growth and cells grew to a higher density.

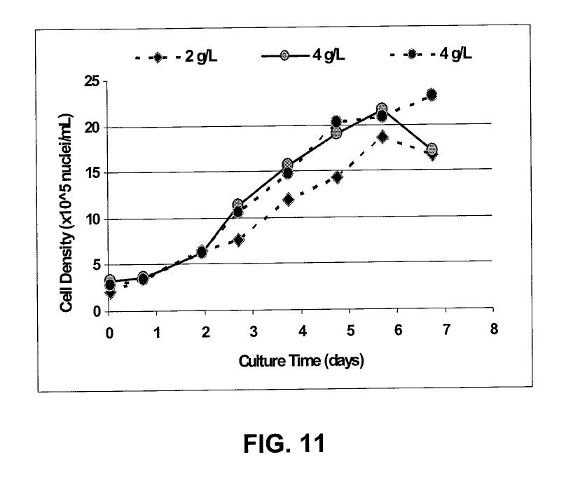

Increasing microcarrier density was examined to see if improvement of Vero cell growth could be obtained. Again, four bioreactor cultures were inoculated with 2e5 cells/mL Vero cells in the growth medium, as described above. The DO was at 50% air saturation, pH was set at 7.1 and temperature was at 37° C. An agitation rate of 125 rpm was used. Two bioreactors contained Cytodex™ 1 at 2 g/L and two other bioreactors at 4 g/L. Cell growth was monitored by taking samples daily from each bioreactor and counting nuclei number in the culture samples. The cell growth data is shown in FIG. 11. Cultures with 4 g/L of Cytodex™ 1 had higher cell density than the culture with 2 g/L of Cytodex™ 1. One bioreactor culture with 2 g/L of Cytodex™ 1 was lost due to equipment failure.

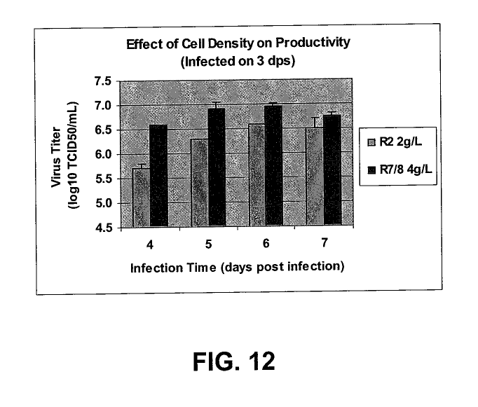

To evaluate whether increasing microcarrier density could result in higher virus titer, 20 mL of cultures were removed from each of the bioreactors on day 3 and transferred to a 125 mL shake flask. The Vero growth medium was replaced with the post-infection medium (SFM4 MegaVir and 4 mM L-Gln). Cells were then infected with RSV dM2-2 virus at MOI of 0.01 and cultured in a shake incubator at 33° C. and 5% CO₂ with shaking at 100 rpm. Virus production was monitored by taking samples from each flask on days 4, 5, 6, and 7. Virus titer was determined by TCID₅₀ assay. As shown in FIG. 12, cultures with 4 g/L of Cytodex™ 1 produced higher virus titer than the culture with 2 g/L of the microcarrier beads. Data for the 4 g/L culture is the average of the two cultures.

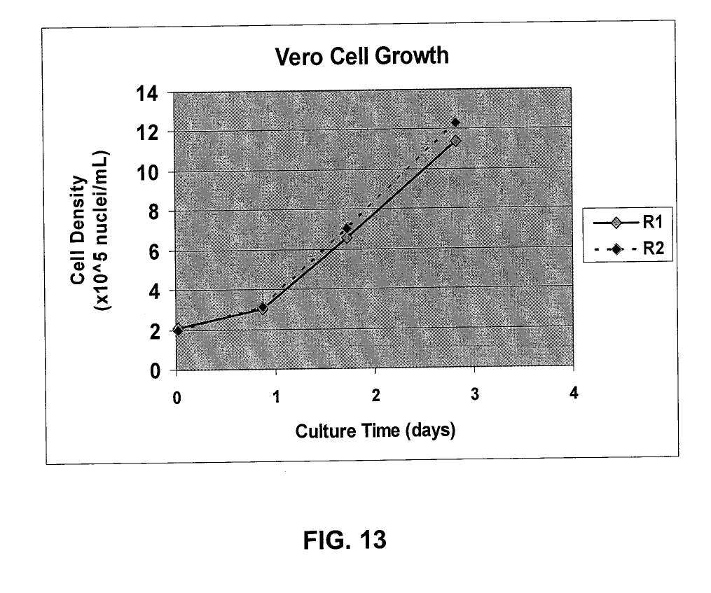

Duplicate bioreactor cultures were inoculated with Vero cells at 2e5 cells/mL in the serum-free growth medium described above and cultured under the optimal condition identified in the above experiments with 4 g/L of Cytodex™ 1, 125 rpm agitation rate, 50% of DO, pH at 7.1 and 37° C. for 3 days. Cell growth was monitored by taking samples daily from each bioreactor and counting nuclei number in the culture samples. The data is shown in FIG. 13.

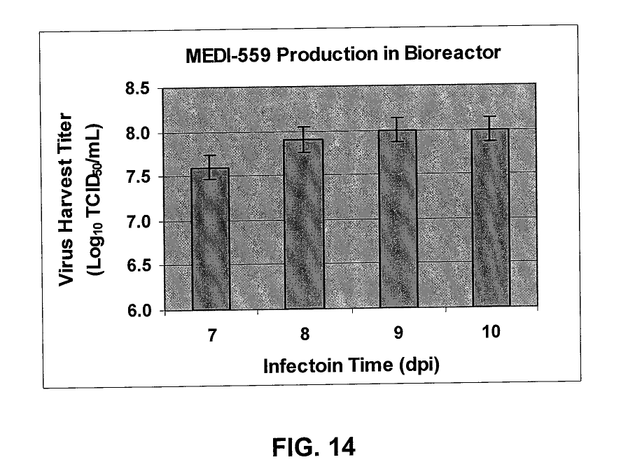

On day 3 of culturing, agitation was stopped to allow the microcarrier beads to settle to the bottom of the bioreactor. Spent growth medium was then removed from the bioreactor while leaving the cells on the microcarrier beads behind and was replaced with equal volume of fresh post-infection medium (SFM4 MegaVir™+4 mM L-Gln). Agitation was resumed at 125 rpm. Temperature of the culture was reduced to 30° C. and the pH was set at 7.0. Cells were then infected with MEDI-559 at the MOI of 0.01 and continued to be cultured at 30° C. for 10 days. Samples were taken from the cultures daily from day 7 to day 10. Virus titers were determined by TCID₅₀ assay. As shown in FIG. 14, the peak productivity of the bioreactor cultures reached 8 logs/mL (log₁₀ TCID₅₀).

6.13 Improving PIV Process Production in Bioreactors

### Cell Line and Culture Maintenance

Vero cell line (ATCC CCL-81) was adapted to serum free growth condition and banked. Cells derived from the working cell bank (WCB 29 Apr. 2003 PN532AC(SF) 03BA01 PJS) were used in all experiments.

The anchorage-dependent Vero cells were routinely seeded at 5×10⁴ cells/mL—in corresponding culture volumes of 35 mL for T-75 flasks, 100 mL for T-225 flasks, and 300 mL for 850 cm² roller bottles (RBs)—and passaged every 3-4 days. For subculturing, spent media were aspirated, cells were rinsed with DPBS and detached from the flasks by treating with appropriate amount of TrypLE solution (Invitrogen, Carlsbad, Calif.) at 37° C. Equal volume of lima bean trypsin inhibitor (Worthington Biochemical Corporation, Lakewood, NJ) was added to neutralize TrypLE activity. All uninfected Vero cells were cultured in VP-SFM (Invitrogen, Carlsbad, Calif.) supplemented with 4 mM L-glutamine and 1% of chemically defined lipid concentrate (CDLC, Invitrogen, Carlsbad, Calif.), T-flask cultures were maintained in 37° C./5% CO₂/95% Rh incubators and RB cultures were placed on a roller bottle apparatus operated at 0.3 rpm in a 37° C. incubator.

### Virus Seed

MEDI-560 is a derivative of cp45, a live, attenuated vaccine candidate for hPIV3 virus. MEDI-560 virus seed stocks were stored at −80° C. and thawed only immediately before use.

### T-flask Experiment

Infection parameters for MEDI-560 production were first screened in T25 flask. T-25 flasks were seeded with 6×10⁵ Vero cells in 12 mL of VP-SFM supplemented with 4 mM L-glutamine and 1% CDLC and maintained in a 37° C./5% CO₂/95% Rh incubator. Infection was done on day 3 post seeding. Cell counts in two T25 flasks were measured by detaching cells with TrypLE solution and counting cells on Vi-Cell Cell Viability Analyzer (Beckman Coulter, Miami, Fla. Model Vi-Cell XR). The average cell count from the two flasks was used to calculate the amount of virus seed to be used for infection based on the multiplicity of infection (MOI) of 0.01 TCID₅₀/cell. Duplicate T25 flasks were infected with MEDI-560 in three infection media (SFM4 MegaVir (Hyclone, Logan, Utah), William's medium E (Lonza), and Ex-Cell Vero (SAFC Biosciences JRH), all supplemented with 4 mM L-glutamine), two temperatures (32° C. and 30° C.) and harvested at three time points (5, 6, and 7 days post infection or dpi) as shown in Table IV. Cultures were maintained in incubator with 5% CO₂/95% Rh. At harvesting, spend culture medium samples were collected from two duplicate flasks from each condition and stabilized with 10% (v/v) of 10× Sucrose-Phosphate (10×SP) buffer. All samples frozen and stored at <−60° C. Infectious virus titer was measured using a TCID50 assay.

TABLE IV
T25 Flask Infection Conditions

| Test | Infection | | Harvesting Time |
|---|---|---|---|
| Condition | Temperature | Infection Media | (days post infection) |
| 1 | 30 C. | SFM4MegaVir | 5 |
| 2 | 30 C. | SFM4MegaVir | 6 |
| 3 | 30 C. | SFM4MegaVir | 7 |
| 4 | 30 C. | William's Medium E | 5 |
| 5 | 30 C. | William's Medium E | 6 |
| 6 | 30 C. | William's Medium E | 7 |
| 7 | 32 C. | SFM4MegaVir | 5 |
| 8 | 32 C. | SFM4MegaVir | 6 |
| 9 | 32 C. | SFM4MegaVir | 7 |
| 10 | 32 C. | William's Medium E | 5 |
| 11 | 32 C. | William's Medium E | 6 |
| 12 | 32 C. | William's Medium E | 7 |
| 13 | 32 C. | Ex-Cell Vero | 5 |
| 14 | 32 C. | Ex-Cell Vero | 6 |
| 15 | 32 C. | Ex-Cell Vero | 7 |

6.14 Vaccine Bioreactor Comparisons

Small scale bioreactor experiments were conducted in 3 L stirred tank bioreactors (Applikon, Foster City, Calif.). Each bioreactor was equipped with an ADI 1030 Bio Controller (Applikon) and an ADI 1035 Bio Console (Applikon). Cytodex™ 1 microcarriers were prepared for use following the manufacturer's instructions.

For pre-infection Vero cell growth, Vero cells harvested from RB cultures were seeded at 2e5 cells/mL density in Vero cell growth medium (VP-SFM supplemented with 4 mM L-Gln and 1% CDLC) with 4 g/L of Cytodex 1 microcarriers or seeded at 1e5 cells/mL with 2 g/L of Cytodex 1 microcarriers (the modified process) in 1.5 to 2 L working culture volume in the 3 L bioreactors. pH was controlled at 7.1±0.05 by the addition of NaOH solution and sparging of CO₂. Temperature was maintained at 37° C. Dissolved oxygen floated from 100% during the early culture time and was maintained at 50% of air saturation as the cell grew, by sparging pure oxygen. Agitation speed was set at 125 rpm.

Infection was done on day 3 or day 5 post seeding. After collecting a sample, all control loops were disabled and microcarrier beads were allowed to settle for ≧30 minutes. Then, a partial medium exchange was performed. Spent growth medium was pumped out and the same volume of fresh infection medium was added through one of the medium addition ports. The extents of medium exchange that ranged from 66 to 90%. During the infection phase, pH was controlled at 7.1±0.05. Temperature was maintained at 30° C. Dissolved oxygen was maintained at 50% of air saturation and agitation was maintained at 125 rpm. The cells were infected at an MOI (Multiplicity of Infection) of 0.01 TCID₅₀/cell.

### Single Use Bioreactor (SUB) Experiments

For Vero cell growth in SUB (50 L SUB Basic Hardware unit (Hyclone, Logan, Utah, Part No. SH3B1744.01), cells were seeded at 1e5 cells/mL density with 2 g/L of Cytodex 1 microcarriers in 30 L of Vero cell growth medium in the SUB. The pH was controlled at 7.1±0.05 by the addition of NaOH solution and sparging of CO₂. Temperature was maintained at 37° C. Dissolved oxygen floated from 100% during the early culture time and was maintained at 50% of air saturation as the cell grew, by sparging pure oxygen. Agitation speed was maintained at 125 rpm during the first day of culturing and reduced to 100 rpm for the remaining time.

Infection was done on day 5 post seeding. After collecting a sample, all control loops were disabled and microcarrier beads were allowed to settle for >30 minutes. Then, a partial medium exchange was performed. Spent growth medium was pumped out and the same volume of fresh infection medium was added through one of the medium addition ports. The extent of medium exchange was 66%. During the infection phase, pH was controlled at 7.1±0.05. Temperature was maintained at 30° C. Dissolved oxygen was maintained at 50% of air saturation and agitation was maintained at 100 rpm. The cells were infected at an MOI (Multiplicity of Infection) of 0.01 TCID₅₀/cell.

### Collection of Infected Culture Samples for Virus Quantification

After sampling the media in infected T-flask and RB cultures, 10% (v/v) sucrose phosphate was added to stabilize the virus samples. After sampling from infected spinner flasks and bioreactors, microcarrier beads in the samples were allowed to settle and the culture supernatants collected were stabilized with 10% (v/v) 10× sucrose phosphate glutamate buffer (10×SPG). All SPG stabilized virus samples were immediately stored at −80° C. until analyses.

Tables V and VI summarize the main differences in the cell growth and infection conditions amongst the three different bioreactor production runs.

TABLE V
Cell Growth Conditions

| Conditions | 3L260307-R9* | 3L120407-R10 | SUB120407 |
|---|---|---|---|
| Vessel | 3 L Applikon | 3 L Applikon | 50 L SUB |
| | bioreactor | bioreactor | |
| Working Culture | 1.5 L | 1.5 L | 30 L |
| Volume | | | |
| Cell Growth Medium | VP-SFM/4 mM L- | VP-SFM/4 mM L- | VP-SFM/4 mM L- |
| | Gln/1% CDLC | Gln/1% CDLC | Gln/1% CDLC |
| Seeding Cell Density | 2e5 | 1e5 | 1e5 |
| (cells/mL) | | | |
| Microcarrier | 4 | 2 | 2 |
| Concentration (g/L) | | | |
| Cells/Microcarrier | 12 | 12 | 12 |
| Agitation (rpm) | 125 | 125 | Day 1 - 100 |
| | | | Days 2 to 3 - 125 |
| | | | Days 4 to 5 - 100 |
| Feeding | Non | Feed glucose and | None |
| | | glutamine to 2 g/L | |
| | | and 2 mM respectively | |
| | | on day 4 post seeding | |

TABLE VI
Virus Infection Conditions

| Conditions | 3L260307-R9 | 3L120407-R10 | SUB120407 |
|---|---|---|---|
| Cell Concentration at | 9.93E5 | 1.25E6 | 8.82E5 |
| Infection (cells/mL) | | | |
| Infection Medium | SFM4MegaVir/ | SFM4MegaVir/ | SFM4MegaVir/ |
| | 4 mM L-Gln | 4 mM L-Gln | 4 mM L-Gln |
| Medium Exchange (%) | 90 | 66 | 66 |
| Point of Infection | 3 | 5 | 5 |
| (days post seeding) | | | |
| Agitation (rpm) | 125 | 125 | 100 |

### Analytical Methods

Cell counts and cell viability from T-flasks and RBs were measured either using a hemacytometer or the Vi-Cell Analyzer operated according to the manufacturer's directions. Cell concentration from bioreactor cultures was determined using a Nucleocounter (New Brunswick Scientific, Edison, N.J., M1293-0000). Concentrations of glucose, lactate, glutamine and ammonium were analyzed with a Bioprofile 400 instrument (Nova Biomedical, Waltham, Mass., Bioprofile 400). The progression of the virus replication was analyzed by measuring the viral infectivity using a 50% tissue culture infective dose (TCID₅₀) assay and results were quantified in log₁₀ TCID₅₀/mL.

### Results

### Infection Parameter Screening in T25 Flask

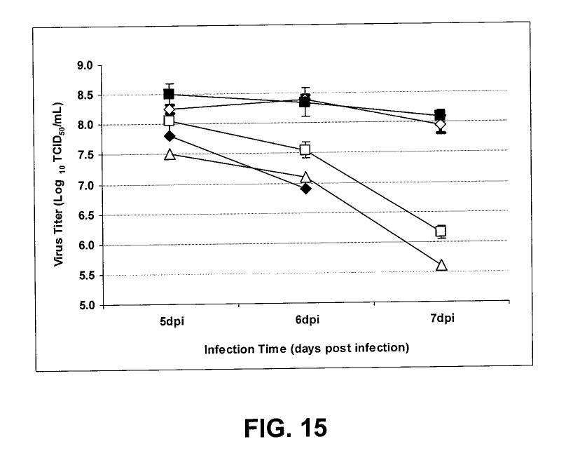

The infectious MEDI-560 titers in the spent culture medium under different infection conditions measured by a TCID50 assay and are shown in FIG. 15. Highest titers were obtained on 5 dpi for all conditions with the exception of when infected in SFM4 MegaVir medium at 30 C, where highest titer was obtained on 6 dpi. Infection conditions of SFM4 MegaVir and William's Medium E at 30° C. produced comparable peak titers of 8.4 and 8.5 logs TCID₅₀/mL respectively. The data shows that MEDI-560 is more stable at 30° C. than at 32° C. in both SFM4 MegaVir and William's Medium E.

The result also indicate that the infection conditions of MOI 0.01, SFM4 MegaVir medium as the infection medium and 30° C. can produce high titers of MEDI-560. The same MOI of 0.01, infection medium SFM4 MegaVir supplemented with 4 mM L-Gln and temperature were used in the bioreactor experiments described below (FIG. 15).

### Vero Cell Growth in Bioreactors

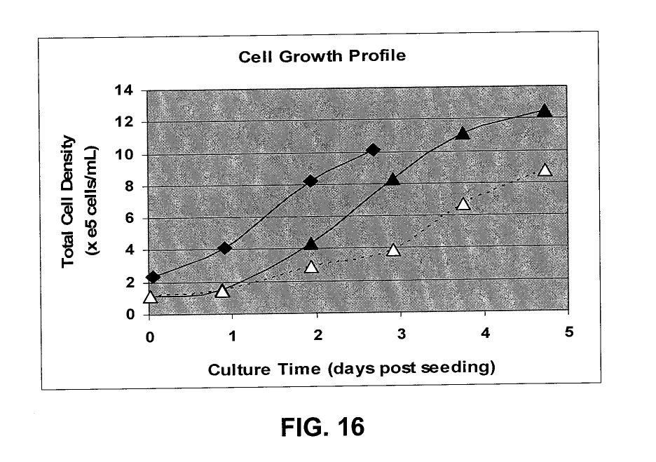

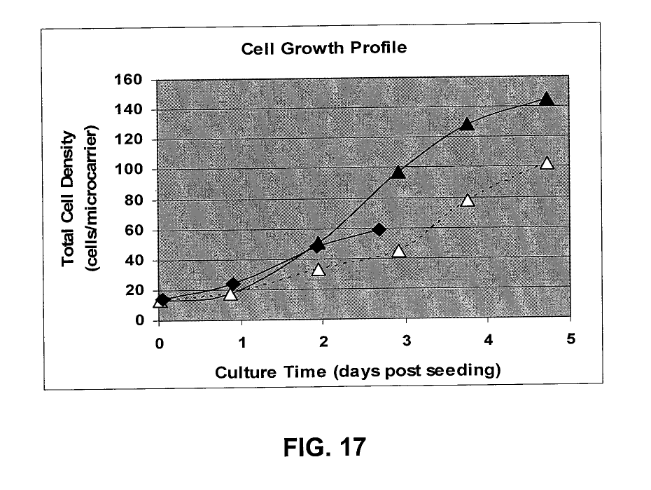

FIG. 16 shows the cell growth profiles of the three bioreactor cultures during the pre-infection phase with cell density measured in cells per milliliter (cells/mL). FIG. 17 shows the cell growth profile with cell density measured in cells per square centimeter (cells/cm²).

As expected, cells in the 1.5 L Applikon bioreactor run, 3L260307-R9 which was seeded at 2e5 cells/mL grew to higher cell density than that in 3L120407-R10 which was seeded at 1e5 cells/mL when cultured in the same period of time, with 3L260307-R9 reaching 1e6 cells/mL and 3L120407-R10 reaching only 8.3e5 cells/mL after 3 days culturing (FIG. 15). However, cells in 3L120407-R10 did reach 1.25e6 cells/mL on day 5 post seeding.

Although 3L120407-R10 was seeded with half the amount of cells used for 3L260307-R9, 1e5 cells/mL vs. 2e5 cells/mL, both reactors were seeded with ˜13 cells per microcarrier as the amount of microcarrier used in 3L120407-R10 was also reduced by half. Cell growth profile for the two 1.5 L bioreactor cultures was similar for the first two days (FIG. 16). However, cell growth in 3L260307-R9 was slower after two days and reached 59 cells/microcarrier on day 3, compared with 96.6 cells/microcarrier in 3L120407-R10. This slow growth observed in 3L260307-R9 may be due to the faster depletion of glucose (FIG. 17).

For SUB 120407, agitation was maintained at 100 rpm during the first day of culturing to allow cells attaching to the microcarrier beads. Agitation was increased to and maintained at 125 rpm from days 2 to 3. However, cell growth in SUB lags behind the 1.5 L Applikon bioreactor culture, 3L120407-R10. In an attempt to improve cell growth, agitation was reduced to and maintained at 100 rpm from days 4 to 5 post seeding. Cells in the SUB culture grew similarly to the 1.5 L bioreactor culture, 3L120407-R10, during the first day. However, cell growth in SUB was considerably slower than the 1.5 L control bioreactor after day 1. It has been shown that agitation rate significantly affect MDCK cell growth in SUB.

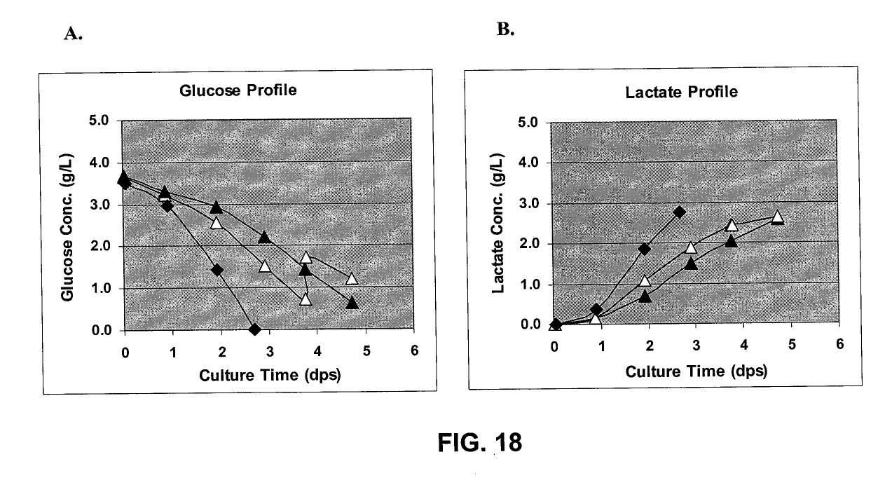

As shown in FIG. 18A-B, glucose was depleted the fastest in 3L260307-R9 as it has the highest cell density. It also produced the highest amount of lactate. 3L120407-R10 produced slightly more lactate than the SUB culture during days 2-4. However, the final lactate concentration on day 5 post seeding was comparable for the two cultures.

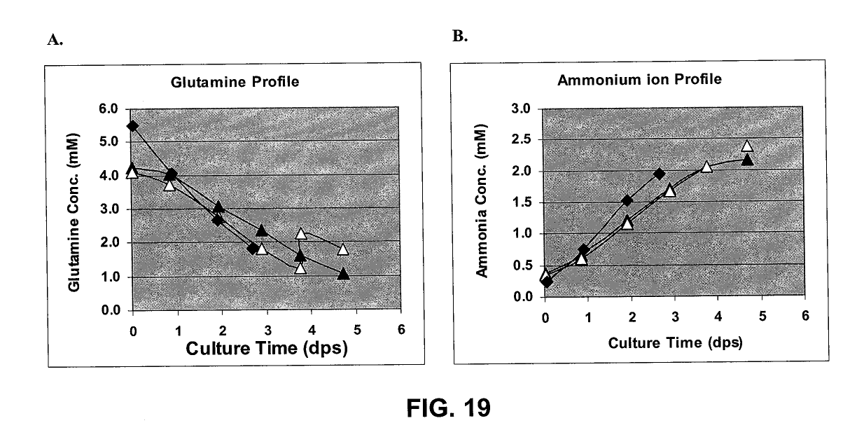

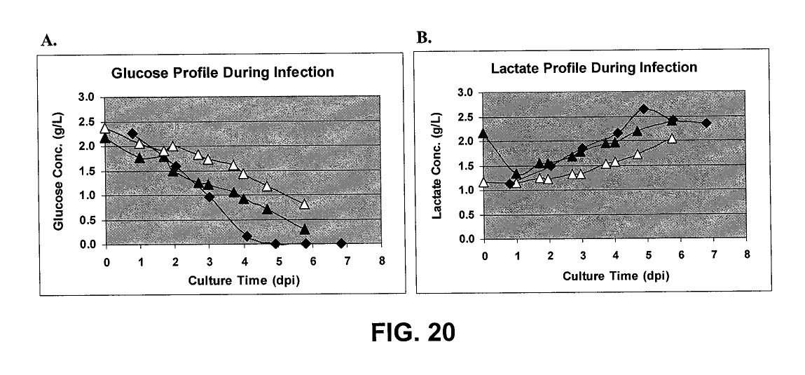

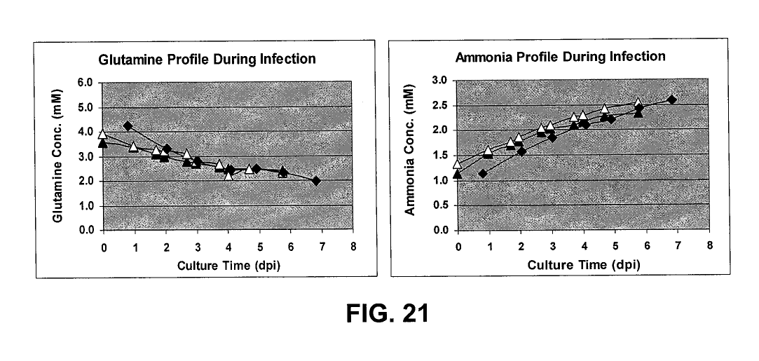

3L260307-R9 had an initial glutamine concentration of 5.6 mM, higher than the calculated concentration of 4 mM (FIG. 19A-B). Consumption of glutamine is slower in the SUB than the other two perhaps because it had the lowest cell density. Ammonium ion production profile for 3L120407-R10 and SUB120407 are very similar. 3L260307-R9 produced more ammonium ion, which is likely due to the fact that it had the highest cell density.

### MEDI-560 Production in Bioreactors

Virus production in the three bioreactor runs was measured using a TCID50 assay and are summarized in Table VII.

TABLE VII
MEDI-560 Titer (log₁₀ TCID₅₀/mL)

| Bioreactor Run ID | 1 dpi | 2 dpi | 3 dpi | 4 dpi | 5 dpi | 6 dpi |
|---|---|---|---|---|---|---|
| 3L120407-R10 | 6.3 | 8.3 ± 0.3 | 8.3 ± 0.1 | 8.4 ± 0.3 | 8.1 ± 0.1 | 8.1 ± 0.1 |
| SUB120407 | 5.8 | 8.1 ± 0.1 | 8.6 ± 0.1 | 8.3 ± 0.1 | 8.4 ± 0.1 | 8.5 ± 0.0 |
| 3L260307-R9 | NA | 8.3 ± 0.3 | 8.5 ± 0.1 | 8.6 ± 0.1 | 8 ± 0.1 | 8.3 ± 0.0 |

The data indicates that MEDI-560 titer peak on 3 dpi in the SUB run and on 4 dpi in the two 1.5 L Applikon bioreactor runs. The peak titers for the three bioreactor runs are comparable.

### Direct Bead-to-bead Transfer Method for Expanding Vero Cells Cultured on Microcarrier in Bioreactors

In order to effectively expand a Vero cell culture, Vero cells need to be detached from the microcarrier beads upon which they are attached and then attach to freshly added microcarrier beads in order to grow. Typically trypsin/EDTA has been used to detach the Vero cells from the microcarrier beads to allow expansion (Sugawara K., et al., Biologicals 2002, 30, 303-314). However, this approach involves removal of culture medium and the utilization of a large amount of trypsin/EDTA. It is cumbersome, costly and increases the risk of contamination.

A direct bead to bead transfer method was developed to expand Vero cell cultures in bioreactors without using trypsin or using a trypsin-like enzyme to detach the Vero cells from the microcarrier beads. Vero cells were allowed to directly migrate from the microcarrier beads they are attached to freshly added beads in order to expand and grow. To test if cells from a expanded bioreactor culture using the direct bead-to-bead transfer method have comparable virus productivity as the cultures seeded with cells from roller bottles, the following comparative study was performed.

Vero cell line (ATCC CCL-81) was adapted to serum free growth condition and banked. Cells derived from the working cell bank (WCB 29 Apr. 2003 PN532AC(SF) 03BA01 PJS) were used in all experiments.

Roller Bottle cultures: The anchorage-dependent Vero cells were seeded at 5×10⁴ cells/mL—in corresponding culture volumes of 35 mL for T-75 flasks, 100 mL for T-225 flasks, and 300 mL for 850 cm² roller bottles (RBs)—and passaged every 3-4 days. For subculturing, spent media was aspirated, cells were rinsed with DPBS and detached from the flasks by treating with appropriate amount of TrypLE solution (Invitrogen, Carlsbad, Calif.) at 37° C. An equal volume of lima bean trypsin inhibitor (Worthington Biochemical Corporation, Lakewood, N.J.) was added to neutralize TrypLE activity. All uninfected Vero cells were cultured in VP-SFM (Invitrogen, Carlsbad, Calif.) supplemented with 4 mM L-glutamine and 1% of chemically defined lipid concentrate (CDLC, Invitrogen, Carlsbad, Calif.), T-flask cultures were maintained in 37° C./5% CO₂/95% Rh incubators and RB cultures were placed on a roller bottle apparatus operated at 0.3 rpm in a 37° C. incubator.

MEDI-560 and MEDI-559 were used in the experiment. The virus seed stocks were stored at −80° C. and thawed only immediately before use.

Direct bead-to-bead bioreactor cultures: Small scale bioreactor experiments were conducted in 3 L stirred tank bioreactors (Applikon, Foster City, Calif.). Each bioreactor was equipped with an ADI 1030 Bio Controller (Applikon) and an ADI 1035 Bio Console (Applikon). Cytodex 1 microcarriers were prepared for use following the manufacturer's instructions.

To start a bioreactor culture, Vero cells harvested from RB cultures were seeded at 2e5 cells/mL density in Vero cell growth medium (VP-SFM supplemented with 4 mM L-Gln and 1% CDLC) with 4 g/L of Cytodex 1 microcarriers or seeded at 1e5 cells/mL with 2 g/L of Cytodex 1 microcarriers in 1.5 to 2 L working culture volume in the 3 L bioreactors. pH was controlled at 7.1±0.05 by the addition of NaOH solution and sparging of CO₂. Temperature was maintained at 37° C. Dissolved oxygen floated from 100% during the early culture time and was maintained at 50% of air saturation as the cell grew, by sparging pure oxygen. Agitation speed was set at 125 rpm.

To test the expansion of Vero cell culture from bioreactor to bioreactor by direct Vero cell migrating from beads to beads without utilizing trypsin, the following experiments were performed. Vero cells were cultured in an Applikon bioreactor in 1.5 L working volume as described above for 3 days when cell density reached ≧1e6 cells/mL. For expansion at 1:1 split ratio, 750 mL to 1 L of the 3 day culture was transferred to a new bioreactor containing an equal volume of fresh growth medium with 4 g/L of fresh Cytodex 1 beads. For expansion at 1:5 split ratio, 300 mL of the Vero cell culture was transferred to a fresh bioreactor containing 1.2 L of fresh growth medium with 4 g/L of fresh Cytodex 1 beads.

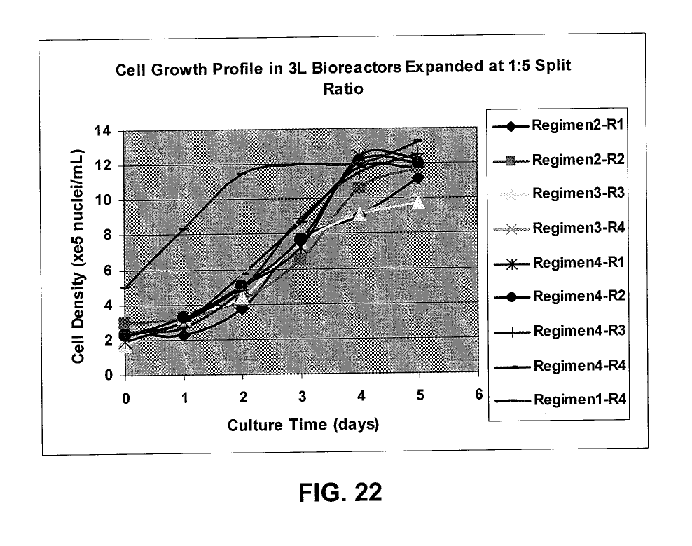

Cells were cultured using the same parameters as described above with the following modification of agitation. Instead of constant agitation at 125 rpm, various intermittent agitation regime were used as shown in Table VIII. Cell growth results in bioreactors expanded with the four different intermittent agitation regime are shown in FIG. 22.

TABLE VIII
Various Intermittent Agitation Regime Tested

| | Cycle 1 | Duration | Cycle 2 | Duration | Cycle 3 | Duration | Comments |
|---|---|---|---|---|---|---|---|
| Regimen | 125 rpm × 5′/ | 5 hrs | Constant | The rest of | NA | NA | 1:1 Split |
| 1 | 0 rpm × 30′ | | 125 rpm | the culture | | | Ratio |
| | | | | time | | | |
| Regimen | 125 rpm × 5′/ | 24 hrs | 125 rpm × 1 hr/ | 24 hrs | Constant | The rest of | 1:5 Split |
| 2 | 0 rpm × 30′ | | 0 rpm × 1 hr | | 125 rpm | the culture | Ratio |
| | | | | | | time | |
| Regimen | 125 rpm × 10′/ | 8 hrs | Constant | The rest of | NA | NA | 1:5 Split |
| 3 | 0 rpm × 50′ | | 125 rpm | the culture | | | Ratio |
| | | | | time | | | |
| Regimen | 125 rpm × 10′/ | 8 hrs | 125 rpm × 1 hr/ | 8 hrs | Constant | The rest of | 1:5 Split |
| 4 | 0 rpm × 50′ | | 0 rpm × 1 hr | | 125 rpm | the culture | Ratio |
| | | | | | | time | |

To test if cells from the expanded bioreactor culture using the direct bead-to-bead transfer method have comparable virus productivity as the cultures seeded with cells from roller bottles, freshly seeded bioreactor cultures and cultures expanded at 1:5 split ratio were infected with MEDI-559 at MOI of 0.01 FFU/cell. During the infection phase, pH was controlled at 7.1±0.05. Temperature was maintained at 30° C. Dissolved oxygen was maintained at 50% of air saturation and agitation was maintained at 125 rpm. As a result, peak virus titers were comparable as shown in Table IX.

TABLE IX
Comparison of Peak MEDI-559 Titers between Freshly Seeded
and Expanded Bioreactor Cultures of Vero Cells

| | Peak Titer (log₁₀ FFU/mL) | SD |
|---|---|---|
| R3 1:5 split | 7.7 | 0.04 |
| R4 1:5 split | 7.7 | 0.03 |
| R7 newly seeded | 7.6 | 0.01 |
| R8 newly seeded | 7.6 | 0.02 |

Vero cells cultured in 3 L bioreactors using the platform process parameters were expanded to 15 L bioreactors in two separate experiments described below in Table X. Cells were transferred from the 3 L bioreactor and expanded to 15 L bioreactor at the split ratio of 1:5. Cells reached ≧1e6 nuclei/mL by day 4 or 5 as seen in cultured expanded in 3 L bioreactors (FIG. 22). Expanded Vero cell cultures in 15 L Applikon bioreactors were infected with MEDI-559 or MEDI-560 at the MOI of 0.01. Peak titers obtained in the 15 L expanded cultures were comparable to that from the 3 L bioreactor cultures (Table X)

TABLE X
Experiments Design and Results of Expansion Vero Cells from 3 L to 15 L Applikon Bioreactors

| | Experiment 1 | | Experiment 2 | |
|---|---|---|---|---|
| Seed bioreactor | 3 L Applikon | | 3 L Applikon | 3 L Applikon |
| Working Volume | 2 L | | 2 L | 2 L |
| Culture Parameter | Platform Process | | Platform Process | |
| Culture Time | 4 days | | 3 days | |
| Final Cell Density | 1.2e6 nuclei/mL | | 1.3e6 nuclei/mL | 1.3e6 nuclei/mL |
| Volume of Culture | 1.6 L | 0.4 L | 2 L | NA |
| Transferred | | | | |
| Reactor Transferred | 15 L Applikon | 3 L control | 15 L Applikon | NA |
| to | | reactor | | |
| Working Volume | 8 L | 2 L | 10 L | NA |
| Expansion Ratio | 1:5 | 1:5 | 1:5 | NA |
| Seeding Cell | 1.8e5 nuclei/mL | 1.2e5 nuclei/mL | 2.1e5 nuclei/mL | NA |
| Density | | | | |
| Agitation Regimen | Regimen 4 with | Regimen 4 | Regimen 4 with | NA |
| | agitation rate | (see Table VIII) | agitation rate | |
| | reduced from 125 to 90 | | reduced from 125 to 90 | |
| | rpm (see Table VIII) | | rpm (see Table VIII) | |
| Culture Time | 4 days | 4 days | 5 days | NA |
| Final Cell Density | 1.2e6 nuclei/mL | 1.1e6 nuclei/mL | 1.1e6 nuclei/mL | NA |
| Virus Infected | MEDI-559 | MEDI-559 | MEDI-560 | MEDI-560 |
| Peak Virus Titer | 7.23 ± 0.05 | 7.39 ± 0.09 | 8.7 log10 | 8.5 log10 |
| | log10 FFU/mL | log10 FFU/mL | TCID50/mL | TCID50/mL |

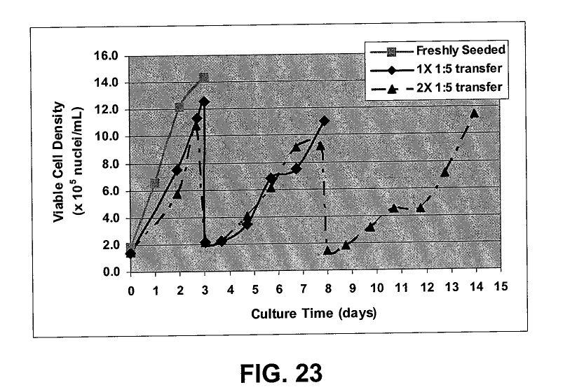

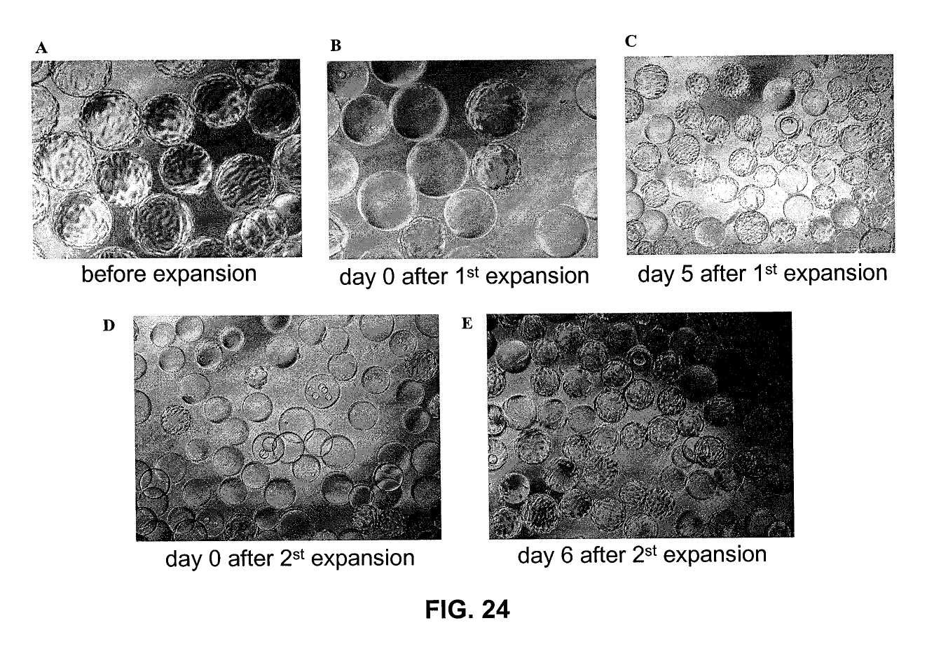

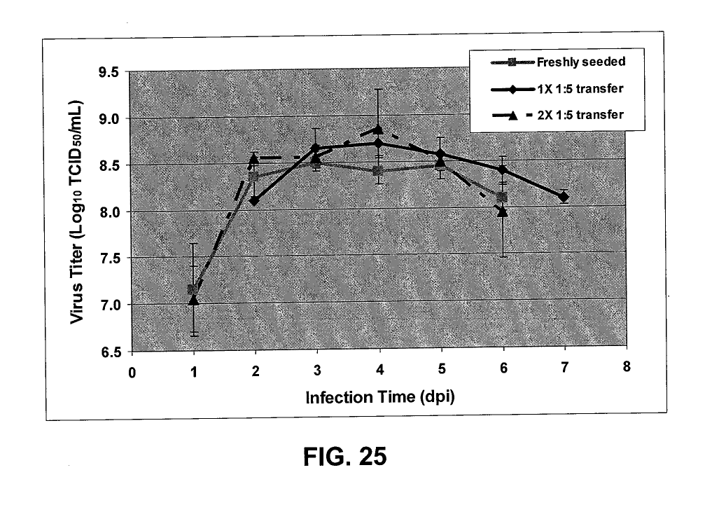

To test if cell distribution homogeneity would be an issue and whether cells derived from multiple expansion retain virus productivity, Vero cells cultures were passaged in 3 L bioreactors 2 consecutive times (2×) at 1:5 split ratio. Cell growth and MEDI-560 production were compared to bioreactor cultures seeded with cells from roller bottle (no expansion) or cultured after one expansion at 1:5 split ratio in bioreactor (1×). Experiment design, cell growth profiles, cell distribution on microcarrier beads, and MEDI-560 productions are shown in Table XI, FIGS. 23 to 25, respectively. Cells reached ≧1e6 nuclei/mL by day 5 (1× expansion) and day 6 after 2 expansions. There was a slight delay in cell growth in expanded cultures. Cell distribution on microcarrier beads in cultures after 1 and 2 expansions are comparable (FIG. 24). All cultures showed comparable MEDI-560 productivity as shown in FIG. 25.

TABLE XI
Experiment Design

| | Freshly Seed | 1× Expansion | 2× Expansion |
|---|---|---|---|
| | Bioreactor | at 1:5 Split | at 1:5 Split |
| | Culture | Ratio | Ratio |
| Seed Culture | 3 L | 3 L | 3 L |
| Vessel | | | |
| Final Culture | 3 L | 15 L | 3 L |
| Vessel | | | |
| Expansion | None | 1× at 1:5 ratio | 2× at 1:5 ratio |
| Virus Infected | MEDI-560 | MEDI-560 | MEDI-560 |

Vero cells expanded using the bead to bead transfer method described above produced comparable RSV vaccines as the Vero cell culture seeded with cells derived from roller bottles.

Various embodiments of the invention have been described. The descriptions and examples are intended to be illustrative of the invention and not limiting. Indeed, it will be apparent to those of skill in the art that modifications may be made to the various embodiments of the invention described without departing from the spirit of the invention or scope of the appended claims set forth below. All references cited herein are hereby incorporated by reference in their entireties. Further, International Patent Application PCT/US07/66037 filed Apr. 5, 2007 and U.S. Provisional Application Nos. 60/862,550 filed Oct. 23, 2006, 60/944,162 filed Jun. 15, 2007, and 60/973,921 filed Sep. 20, 2007 are also each incorporated herein by reference in their entireties.

1-50. (canceled)

51. A Vero cell culture, comprising Vero cells infected with a virus in a cell culture medium substantially free of serum, wherein said culture yields a viral titer of at least 7.0 log₁₀ TCID₅₀/ml.

52. The culture of claim 51, wherein said cell culture medium comprises a glucose concentration of about 0.5 to about 2.5 g/L; and/or a lactate concentration of about 1.0 to about 2.0 g/L; and/or a glutamine concentration of about 2.0 to about 4.0 g/L; and/or an ammonium ion concentration of about 1.25 to about 2.5 mM.

53. The culture of claim 51, wherein said culture yields a viral titer of at least 8.0 log₁₀ TCID₅₀/ml.

54. The culture of claim 51, wherein the virus is a negative strand RNA virus.

55. The culture of claim 54, wherein the negative strand RNA virus is a paramyxovirus that is parainfluenza virus or a respiratory syncytial virus or a metapneumovirus.

56. The culture of claim 54, wherein the negative strand RNA virus is a recombinant virus.

57. A method for propagating a virus in Vero cells comprising:
a. culturing the Vero cells in a bioreactor at a first temperature, comprising seeding a cell culture medium containing chemically-defined lipid concentrate (CDLC) and microcarriers with the Vero cells;
b. infecting the Vero cells cultured in step (a) at a second temperature at a multiplicity of infection of about 0.001 to about 0.10, wherein said second temperature is lower than said first temperature; and
c. recovering the virus from the cell culture of step (c), wherein said recovered virus yields a viral titer of at least 7.0 log₁₀ TCID₅₀/ml.

58. The method of claim 57, wherein the CDLC is added to a concentration of 1% v/v.

59. The method of claim 57, wherein in the cell culture medium is a serum free medium selected from the group consisting of OptiPRO™ SFM, VP-SFM, SFM4 MegaVir™, Ex-Cell Vero™, or WME.

60. The method of claim 57, wherein the chemically defined lipid concentrate comprises one or more of Pluronic F-68, Ethyl Alcohol, Cholesterol, Tween 80, DL-alpha-Tocopherol Acetate, Stearic Acid, Myristic Acid, Oleic Acid, Linoleic Acid, Palmitic Acid, Palmitoleic Acid, Arachidonic Acid, and Linolenic Acid.

61. The method of claim 57, wherein the chemically defined lipid concentrate comprises one or more of 100,000 mg/L of Pluronic F-68, 100,00 mg/L of Ethyl Alcohol, 220 mg/L of Cholesterol, 2,200 mg/L of Tween 80, 70 mg/L of DL-alpha-Tocopherol Acetate, 10 mg/L of Stearic Acid, 10 mg/L of Myristic Acid, 10 mg/L of Oleic Acid, 10 mg/L of Linoleic Acid, 10 mg/L of Palmitic Acid, 10 mg/L of Palmitoleic Acid, 2 mg/L of Arachidonic Acid, and 10 mg/L of Linolenic Acid.

62. The method of claim 57, wherein the first temperature is between about 36° C. and about 38° C.

63. The method of claim 57, wherein the second temperature is between about 30° C. and about 33° C.

64. The method of claim 57, wherein the microcarrier concentration is between about 1 to about 4 g/L.

65. The method of claim 57, wherein after step (a) but prior to step (b) about 50% to about 90% of the cell culture medium is exchanged.

66. The method of claim 57, wherein the cell culture medium is exchanged for a cell culture media having the same composition or a cell culture media having a different composition.

67. The method of claim 57, wherein the multiplicity of infection is about 0.01.

68. The method of claim 57, wherein the virus is negative strand RNA virus.

69. The method of claim 68, wherein the negative strand RNA virus is a paramyxovirus that is a parainfluenza virus or a respiratory syncytial virus or a metapneumovirus.

70. The method of claim 68, wherein the negative strand RNA virus is a recombinant virus.

71. The method of claim 57, wherein the recovered virus yields a viral titer of at least 8.0 log₁₀ TCID₅₀/ml or at least 9.0 log₁₀ TCID₅₀/ml.

72. The method of claim 57, wherein said method produces at least 2 million, at least 9 million, at least 12 million, or at least 120 million vaccine doses per 30 L virus harvest batch.
